(19) United States
(12) Patent Application Publication
(10) Pub. No.: US 2023/0218618 A1
(43) Pub. Date: Jul. 13, 2023

(54) ADMINISTRATION OF SUMO-ACTIVATING ENZYME INHIBITOR AND ANTI-CD38 ANTIBODIES

(71) Applicant: MILLENNIUM PHARMACEUTICALS, INC., Cambridge, MA (US)

(72) Inventors: Dennis HUSZAR, Watertown, MA (US); Akito NAKAMURA, Brookline, MA (US)

(21) Appl. No.: 17/998,741

(22) PCT Filed: May 14, 2021
(86) PCT No.: PCT/US2021/032468

§ 371 Date: Nov. 14, 2022

Related U.S. Application Data
(60) Provisional application No. 63/025,554, filed on May 15, 2020.
(60) Provisional application No. 63/136,957, filed on Jan. 13, 2021.
(60) Provisional application No. 63/140,398, filed on Jan. 22, 2021.

(51) Int. Cl. A61K 31/506; C07K 16/28; A61P 35/02

(52) U.S. Cl. CPC A61K 31/506; A61P 35/02; C07K 16/2896; A61K 2039/545

(57) ABSTRACT

The present disclosure provides methods, pharmaceutical compositions, and kits for treating cancer or autoimmune disease in patients in need thereof. The methods comprise administering to a patient in need a small ubiquitin-like modifier (SUMO) activating enzyme (SAE) inhibitor, such as [(1R,2S,4R)-4-{[5-({4-[(1R)-7-chloro-1,2,3,4-tetrahydroisoquinolin-1- yl]-5-methyl-2-thienyl}carbonyl)pyrimidin-4-yl]amino}-2-hydroxy-cyclopentyl]methyl sulfamate (Compound I-263a) or a pharmaceutically acceptable salt, in combination with one or more anti-CD38 antibodies. Also provided are medicaments for use in treating cancer or autoimmune disease.

### FIELD

The present disclosure relates to methods of treating cancer and autoimmune diseases. In particular, the present disclosure provides methods for treating various cancers and autoimmune diseases by administering a small ubiquitin-like modifier (SUMO) activating enzyme (SAE) inhibitor in combination with one or more anti-CD38 antibodies.

### BACKGROUND

In 2012, there were an estimated 14 million cases of cancer diagnosed worldwide and about 8.2 million deaths. The global cancer burden is growing at an alarming pace; in 2030 alone, about 21.3 million new cancer cases and 13.1 million cancer deaths are expected to occur, simply due to the growth and aging of the population. Cancer is the second most common cause of death in the US, exceeded only by heart disease, accounting for nearly 1 of every 4 deaths. The National Cancer Institute estimates that approximately 14.5 million Americans with a history of cancer were alive in 2014. Some of these individuals were cancer free, while others still had evidence of cancer and may have been undergoing treatment. Although medical advances have improved cancer survival rates, there is a continuing need for new and more effective treatment.

Cancer treatments have mainly relied on the combination of surgery, radiotherapy, and/or cytotoxic chemotherapies. Within the last decade, however, targeted cancer therapies have opened a new era in the field of oncology. Targeted cancer therapies are drugs designed to interfere with specific molecules necessary for tumor growth and progression, and can include small molecules and larger chemical entities, such as monoclonal antibodies (mAbs).

CD38 is a multifunctional protein having function in receptor-mediated adhesion and signaling as well as mediating calcium mobilization via its ecto-enzymatic activity, catalyzing formation of cyclic ADP-ribose (cADPR) and ADPR. CD38 mediates cytokine secretion and activation and proliferation of lymphocytes (Funaro et al., J. Immunolog 145:2390-6, 1990; Terhorst et al., Cell 771-80, 1981; Guse et al., Nature 398:70-3, 1999). CD38 is expressed in a variety of malignant hematological diseases, including multiple myeloma, leukemias and lymphomas such as B-cell chronic lymphocytic leukemia, T-and B-cell acute lymphocytic leukemia, Waldenstrom macroglobulinemia, primary systemic amyloidosis, mantle-cell lymphoma, pro-lymphocytic/myelocytic leukemia, acute myeloid leukemia, chronic myeloid leukemia, follicular lymphoma, Burkitt’s lymphoma, large granular lymphocytic (LGL) leukemia, NK-cell leukemia and plasma-cell leukemia.

Multiple myeloma is a hematological malignancy characterized by the expansion of malignant plasma cells in the bone marrow. (Saltarella et al., Cell, 9, 167-180, 2020). Despite the availability of new anti- multiple myeloma agents, i.e., proteasome inhibitors and immunomodulatory drugs, multiple myeloma remains an incurable disease. (Fairfield et al., Ann. N. Y. Acad. Sci., 1364, 32-51, 2016; Solimando et al., J. Clin. Med., 8, 997, 2019). A real-world retrospective study demonstrated that proteasome inhibitors and immunomodulatory drugs refractory patients show a median overall survival of approximately eight months. (Usmani et al., Oncologist, 21, 1355-1361, 2016). Daratumumab, a human-specific anti-CD38 IgG1 monoclonal antibody, is the first-in-class human-specific biologic approved for the treatment of multiple myeloma. (Touzeau et al., Expert Opin. Biol. Ther., 17, 887-893, 2017). However, despite the well-established clinical efficacy of daratumumab, approximately 60% of patients do not achieve a partial response, and ultimately all patients undergo progression. (Nooka et al., Cancer, 125, 2991-3000, 2019). Thus, novel and effective therapies are needed to address this high unmet medical need.

Non-Hodgkin lymphoma (NHL) is among the most common cancers in the United States and Europe with more than 70,000 and 93,000 new cases diagnosed every year, respectively. Siegel R.L., et al., CA Cancer J. Clin. 68(1):7-30 (2018); Ferlay J., et al., Eur. J. Cancer 103:356-87 (2018). NHL is a heterogeneous group of malignancies with varying clinical characteristics that are optimally managed through a range of different treatment modalities. The spectrum of NHL includes more indolent variants such as follicular and marginal zone lymphomas, to more aggressive subtypes such as diffuse large B-cell lymphoma (DLBCL). While systemic chemotherapy is a mainstay of treatment for most NHL variants, antitumor directed monoclonal antibodies have an important role in the treatment of this disease. Oflazoglu E., et al., MAbs 2(1): 14-9 (2010). Monoclonal antibodies such as daratumumab, which targets the B-cell antigen CD38, are now part of the standard treatment regimens for many B-cell NHLs. Keating GM, Drugs 70(11):1445-76 (2010). However, once NHL becomes refractory to standard chemotherapy and antibody-based therapies, the overall prognosis is poor, with limited long-term survival. Thus, novel and effective therapies are needed to address this high unmet medical need.

Small ubiquitin-like modifier (SUMO) activating enzyme (SAE) inhibitors are examples of small molecules that can be used for targeted therapies. SUMO is a member of the ubiquitin-like protein (Ubl) family that covalently conjugate to cellular proteins in a manner similar to Ub-conjugation (Kerscher, O. et al., Annu Rev Cell Dev Biol. 22:159-80 (2006)). Mammalian cells express three major isoforms: SUMO1, SUMO2, and SUMO3. SUMO2 and SUMO3 share ~95% amino acid sequence homology but have ~45% sequence homology with SUMO1 (Kamitani, T., et al., J Biol Chem. 273(18):11349-53 (1998)). SUMO proteins can conjugate to a single lysine residue of a protein (monosumoylation) or to a second SUMO protein that is already conjugated to a protein forming a SUMO chain (polysumoylation). Only SUMO2/3 can form such chains because they possess internal consensus SUMO modification sites (Tatham, M. H., et al., J Biol Chem. 276(38):35368-74 (2001)). An additional isoform, SUMO4, is found in kidney, lymph node and spleen cells, but it is not known whether SUMO4 can conjugate to cellular proteins.

SUMO1, SUMO2 and SUMO3 are activated in an ATP-dependent manner by SAE (see, for example, U.S. Pat. Application Publication No. 2010/0160177 A1 (FIG. 1B) and U.S. Pat. 9,434,765 B2(FIG. 2)). SAE is a heterodimer that consists of SAE1 (SUMO-activating enzyme subunit 1) and SAE2 (UBA2). SAE, like other E1 activating enzymes, uses ATP to adenylate the C-terminal glycine residue of SUMO. In a second step, a thioester intermediate is then formed between the C-terminal glycine of SUMO and a cysteine residue in SAE2. Next, SUMO is transferred from the E1 to the cysteine residue of the SUMO conjugating enzyme (E2), UBC9. Unlike the Ub pathway that contains many E2 enzymes, Ubc9 is currently the only known conjugating enzyme for SUMO and functions with SUMO1, SUMO2, and SUMO3 proteins. SUMO proteins then conjugate to the target protein, either directly or in conjunction with an E3 ligase, through isopeptide bond formation with the epsilon amino group of a lysine side chain on a target protein. Several SUMO E3 ligases, including PIAS (protein inhibitor of activated signal transducer and activator of transcription protein) proteins and Ran-binding protein 2 (RanBP2), and polycomb 2 (Pc2), have been identified (Johnson, E. S., and Gupta, A. A, Cell. 106(6):735-44 (2001); Pichler, A., etal., Cell. 108(1):109-20 (2002); Kagey, M. H., et al., Cell. 113(1): 127-37 (2003)). Once attached to cellular targets, SUMO modulates the function, subcellular localization, complex formation and/or stability of substrate proteins (Müller, S., et al., Nat Rev Mol Cell Biol. 2(3):202-10 (2001)). SUMO-conjugation is reversible through the action of de-sumoylating enzymes called SENPs (Hay, R. T., Trends Cell Biol. 17(8):370-6 (2007)) and the SUMO proteins can then participate in additional conjugation cycles.

SAE-initiated SUMO-conjugation plays a major role in regulating diverse cellular processes, including cell cycle regulation, transcriptional regulation, cellular protein targeting, maintenance of genome integrity, chromosome segregation, and protein stability (Hay, R. T., Mol Cell. 18(1):1-12 (2005); Gill, G., Genes Dev. 18(17):2046-59 (2004)). For example, SUMO-conjugation causes changes in the subcellular localization of RanGAP1 by targeting it to the nuclear pore complex (Mahajan, R., et al., Cell. 88(1):97-1070 (1997)). Sumoylation counteracts ubiquitination and subsequently blocks the degradation of IκB, thereby negatively regulating NF-xB activation (Desterro, J. M., et al., Mol Cell. 2(2):233-9 (1998)). Sumoylation has been reported to play an important role in transcription exhibiting both repressive and stimulatory effects. Many of the transcriptional nodes that are modulated play important roles in cancer. For example, sumoylation stimulates the transcriptional activities of transcription factors such as p53 and HSF2 (Rodriguez, M. S., et al., EMBO J. 18(22):6455-61 (1999); Goodson, M. L., et al., J Biol Chem. 276(21):18513-8 (2001)). In contrast, SUMO-conjugation represses the transcriptional activities of transcription factors such as LEF (Sachdev, S., et al., Genes Dev. 15(23):3088-103 (2001)) and c-Myb (Bies, J., et al., J Biol Chem. 277(11):8999-9009 (2002)). SUMOylation has also been shown to regulate the production of Type I interferons (Crowl, J.T. and Stetson, D.B. PNAS 115(26):6798-6803 (2018); Decque, A., et al., Nature Immunology 17(2):140-149 (2016)). Thus, SUMO-conjugation controls gene expression and growth control pathways that are important for cancer cell survival.

Altered expression of SAE pathway components have been noted in a variety of cancer types: (Moschos, S. J., et al., Hum Pathol. 41(9):1286-980 (2010)); including multiple myeloma (Driscoll, J. J., et al., Blood. 115(14):2827-34 (2010)); and breast cancer (Chen, S. F., et al., Chin J Cancer. 30(9):638-44 (2011)). In addition, preclinical studies indicate that Myc-driven cancers may be especially sensitive to SAE inhibition (Kessler, J. D., et al., Science. 335(6066):348-53 (2012); Hoellein, A., et al., Blood. 124(13):2081-90 (2014)). Since SUMO-conjugation regulates essential cellular functions that contribute to the growth and survival of tumor cells, targeting SAE could represent an approach to treat proliferative disorders such as cancer. (He, X., et al., Nature Chemical Biology. 13: 1164-1171 (2017)). Thus, some cancers may be SAE-mediated disorders.

SAE inhibitors may also be applicable for the treatment of other diseases and conditions outside of oncology. For example, SUMO modifies proteins that play important roles in neurodegenerative diseases (Steffan, J. S., et al., Science. 304(5667):100-4 (2004); Dorval, V., and Fraser, P. E., J Biol Chem. 281(15):9919-24 (2006); Ballatore, C., et al., Nat Rev Neurosci. 8(9):663-72(2007)). Sumoylation also has been reported to play an important role in pathogenic viral infection, inflammation and cardiac function (Lee, H. R., et al., J Virol. 78(12):6527-42 (2004); Liu, B., and Shuai, K., Mol Cell. 35(6):731-2 (2009); Wang, J., and Schwartz, R. J., Circ Res.107(1):19-29 (2010)).

In addition to small molecules, targeted therapies can include monoclonal antibodies. For example, among the many known monoclonal antibody targeted therapies are monoclonal antibodies to CD38 (e.g., daratumumab/Darzalex® for treating multiple myeloma).

New combinations of therapeutic agents that provide a beneficial effect in the treatment of cancers are desirable in order to prolong patient’s lives while maintaining a high quality of life. New combinations may provide an increased benefit as compared to each of the agents alone. In particular, combined treatment regimens may be helpful for patients suffering from disease conditions including proliferative disorders, autoimmune diseases, inflammatory diseases, fibrotic diseases and kidney diseases, and could potentially even decrease the rate of relapse or overcome the resistance to a particular anticancer agent sometimes seen in these patients. This is especially true in the case where the cancers may be resistant or refractory to currently available therapeutic regimens.

Thus, there is a need for new cancer treatment regimens, including combination therapies.

### SUMMARY

In one aspect, the present disclosure relates to methods of treating a disorder, wherein the disorder is cancer or autoimmune disease, comprising administering an SAE inhibitor and an anti-CD38 antibody in combination to a subject in need of such treatment.

In one aspect, the present disclosure relates to methods of treating a disorder, wherein the disorder is cancer or autoimmune disease, comprising administering to a patient in need of said treating a combination of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and an anti-CD38 antibody. Compound I-263a is also referred to herein as TAK-981.

In some embodiments, the anti-CD38 antibody is a selected from the group consisting of daratumumab, isatuximab, MOR03087 (also known as MOR202); SG303, mAb024, mAb003, and mezagitamab (also known as TAK-079, and alternately referred to as AB79 herein).

In some embodiments, the anti-CD38 antibody is mezagitamab.

In some embodiments, the anti-CD38 antibody is daratumumab. In some embodiments, the daratumumab is administered in an injectable formulation that comprises daratumumab and hyaluronidase. In some embodiments, the hyaluronidase is hyaluronidase-fihj.

In some embodiments, the anti-CD38 antibody is isatuximab.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered orally.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered intravenously.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered subcutaneously.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered by intravenous infusion.

In some embodiments, the anti-CD38 antibody is administered intravenously.

In some embodiments, the anti-CD38 antibody is administered by intravenous infusion.

In some embodiments, the anti-CD38 antibody is administered by subcutaneous injection.

In some embodiments, the anti-CD38 antibody is administered subcutaneously.

In some embodiments, the disorder is cancer.

In some embodiments, the disorder is CD38 positive cancer.

In some embodiments, the disorder is hematological malignancy.

In some embodiments, the disorder is multiple myeloma.

In some embodiments, the disorder is CD38 positive multiple myeloma.

In some embodiments, the disorder is CD38 positive relapsed or refractory multiple myeloma.

In some embodiments, the disorder is lymphoma or leukemia.

In some embodiments, the disorder is non-Hodgkin lymphoma. In some embodiments the subject suffers from relapsed or refractory non-Hodkins lymphoma. In some embodiments the disorder is follicular lymphoma (FL), mantle cell lymphoma (MCL), Diffuse large B-cell lymphoma (DLBCL), or Burkitt lymphoma.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered once every two weeks, once every week, twice a week, three times a week, or daily.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered twice a week.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered once every week.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered once every two weeks.

In some embodiments, the [(1R,2S,4R)-4-{[5-(4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered once every four weeks.

In some embodiments, the [(1R,2S,4R)-4-[5-{(4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered once every month.

In some embodiments, the anti-CD38 antibody is administered once every eight weeks, once every four weeks, once every three weeks, once every two weeks, once every week, twice a week, three times a week, or daily.

In some embodiments, the anti-CD38 antibody is administered once every month.

In some embodiments, the anti-CD38 antibody is administered once every four weeks.

In some embodiments, the anti-CD38 antibody is administered once every three weeks.

In some embodiments, the anti-CD38 antibody is administered once every two weeks.

In some embodiments, the anti-CD38 antibody is administered once every week.

In some embodiments, the anti-CD38 antibody is administered twice a week.

In some embodiments, the anti-CD38 antibody is administered once every eight weeks.

In some embodiments, the treatment cycle is 14 days, 21 days, 28 days, or 35 days.

In some embodiments, the anti-CD38 antibody is administered on days 1, 4, 8, and 11 of a 14 days cycle.

In some embodiments, the anti-CD38 antibody is administered on days 0, 3, 7, and 10 of a 14 day cycle.

In some embodiments, the anti-CD38 antibody is administered on days 1, 4, 8, 11, 15, 18, 22 and 25 of a 28 day cycle.

In some embodiments, the anti-CD38 antibody is administered on days 1, 4, 8, 11, 15, and 18 of a 21 day cycle.

In some embodiments, the anti-CD38 antibody is administered on days 1, 8, 15, and 22 of a 28 day cycle.

In some embodiments, the anti-CD38 antibody is administered on days 1 and 15 of a 28 day cycle.

In some embodiments, the anti-CD38 antibody is administered on days 1, 4, 8, 11, and 15 of a 28 day cycle.

In some embodiments, the anti-CD38 antibody is administered on days 1 and 15 of a 28 day cycle.

In some embodiments, the anti-CD38 antibody is administered on day 1 of a 28 day cycle.

In some embodiments, the anti-CD38 antibody is administered once weekly in cycles 1 and 2, followed by every 2 weeks in cycles 3 through 6, then monthly.

In some embodiments, the anti-CD38 antibody is administered once weekly in cycles 1 and 2 of a 28-day cycle, followed by every 2 weeks in cycles 3 through 6, then monthly.

In some embodiments, the anti-CD38 antibody is administered on days 1, 4, 8, 11, 15, 18, 22, 25, 29 and 33 of a 35 day cycle.

In some embodiments, the anti-CD38 antibody is administered in a loading dose or induction schedule for anywhere from 1 to 54 weeks followed by a maintenance dose or consolidation schedule thereafter.

In some embodiments, 45 mg, 135 mg, 300 mg, 500 mg, 600 mg, 700 mg, 900 mg, 1100 mg, 1200 mg, or 1800 mg of the anti-CD38 antibody is administered. In some embodiments, 600 mg of the anti-CD38 antibody is administered. In some embodiments, 1800 mg of the anti-CD38 antibody is administered.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and the anti-CD38 antibody are administered simultaneously once every eight weeks, once every four weeks, once every three weeks, once every two weeks, once every week, twice a week, three times a week, or daily.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1, 4, 8, and 11 of a 14 days cycle.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 0, 3, 7, and 10 of a 14 day cycle.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1, 8, 15, and 22 of a 28 day cycle.

In some embodiments, the [(1R,2S,4R)-4-{[5-(4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1, 4, 8, and 11 of a 21 day cycle.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1 and 8 of a 21 day cycle.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1, 4, 8, 11, and 15 of a 28 day cycle.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1, 4, 8, 11, and 15 of a 28 day cycle in cycles 1 and 2 followed by every 2 weeks in cycles 3 through 6, then monthly.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1, 8, 15, and 22 of a 28 day cycle.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1, 8, 15, and 22 of a 28 day cycle in cycles 1 and 2 followed by every 2 weeks in cycles 3 through 6, then monthly.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1 and 15 of a 28 day cycle.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on day 1 of a 28 day cycle.

In some embodiments, 3 mg, 6 mg, 10 mg, 15 mg, 25 mg, 40 mg, 60 mg, 90 mg, 120 mg, 160 mg, 200 mg, or 250 mg of the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered.

In some embodiments, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, or 250 mg of the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered at 15 mg/kg of the patient’s body weight.

In one aspect, the present disclosure relates to a kit comprising a medicament for use in treating cancer or autoimmune disease in a subject in need of such treatment. The kit comprises a medicament comprising an SAE inhibitor, and instructions for administering the SAE inhibitor and the one or more anti-CD38 antibodies; or the kit comprises a medicament comprising the one or more anti-CD38 antibodies, and instructions for administering the one or more anti-CD38 antibodies and an SAE inhibitor. The kit can contain both a medicament comprising an SAE inhibitor and a medicament comprising one or more anti-CD38 antibodies, and instructions for administering the SAE inhibitor and the one or more anti-CD38 antibodies. The kit can also comprise one or more additional therapeutic agents.

In one aspect, the present disclosure relates to a medicament for use in treating cancer or autoimmune disease in a subject in need of such treatment. The medicament comprises an SAE inhibitor and one or more anti-CD38 antibodies. The medicament can also comprise one or more additional therapeutic agents.

In some embodiments, the additional therapeutic agent is lenalidomide, dexamethasone, bortezomib, melphalan, prednisone, pomalidomide, or combinations thereof. In some embodiments, the additional therapeutic agent is lenalidomide and dexamethasone. In some embodiments, the additional therapeutic agent is bortezomib. In some embodiments, the additional therapeutic agent is melphalan and prednisone. In some embodiments, the additional therapeutic agent is pomalidomide and dexamethasone.

It is to be understood that both the foregoing summary and the following detailed description are exemplary and explanatory only, and are not restrictive of the invention as claimed.

### BRIEF DESCRIPTION OF THE FIGURES

FIG. 1 shows a plot of average tumor growth in a Daudi xenograft human Burkitt’s lymphoma subcutaneous tumor model following administration of vehicle, Compound I-263a alone, daratumumab alone, or a combination of I-263a and daratumumab to mice.

FIG. 2a shows a plot of average tumor growth in a A20-hCD38 mouse B-cell lymphoma line expressing human CD38 tumor model following administration of vehicle, Compound I-263a alone, daratumumab alone, or a combination of I-263a and daratumumab to mice.

FIG. 2b shows Kaplan-Meier survival plots in a A20-hCD38 mouse B-cell lymphoma line expressing human CD38 tumor model following administration of vehicle, Compound I-263a alone, daratumumab alone, or a combination of I-263a and daratumumab to mice.

FIG. 3a shows a plot of average tumor growth in a LP-1 xenograft human multiple myeloma subcutaneous tumor model following administration of vehicle, Compound I-263a alone, daratumumab alone, or a combination of I-263a and daratumumab to mice.

FIG. 3b shows Kaplan-Meier survival plots in a LP-1 xenograft human multiple myeloma subcutaneous tumor model following administration of vehicle, Compound I-263a alone, daratumumab alone, or a combination of I-263a and daratumumab to mice.

FIG. 4 shows a plot of average tumor growth in a MOLP-8 xenograft human multiple myeloma subcutaneous tumor model following administration of vehicle, Compound I-263a alone, daratumumab alone, or a combination of I-263a and daratumumab to mice.

FIG. 5 shows a plot of average tumor growth in a NCI-H929 xenograft human multiple myeloma subcutaneous tumor model following administration of vehicle, Compound I-263a alone, daratumumab alone, or a combination of I-263a and daratumumab to mice.

FIG. 6 shows a plot of average tumor growth in a RPMI-8226 human multiple myeloma subcutaneous tumor model following administration of vehicle, Compound I-263a alone, daratumumab alone, or a combination of I-263a and daratumumab to mice.

FIG. 7 shows a plot of average tumor growth in a Daudi xenograft human Burkitt’s lymphoma subcutaneous tumor model following administration of vehicle, Compound I-263a alone, daratumumab alone, AB79 alone, a combination of I-263a and daratumumab, or a combination of I-263a and AB79 to mice.

### DETAILED DESCRIPTION

### Definitions and Abbreviations

To facilitate an understanding of the present disclosure, a number of abbreviations, terms, and phrases are defined below.

| | |
|---|---|
| SUMO | small ubiquitin-like modifier |
| SAE | SUMO-activating enzyme |
| BIW | twice weekly |
| QW | once weekly |
| Q2W | once every 2 weeks |
| Mg | milligram |
| ML | milliliter |
| Mg/kg | milligram per kilogram of the patient’s body weight |
| H | hour |
| Min | minutes |
| mm³ | cubic millimeter |
| BSA | body surface areas |
| HPβCD | 2-hydroxypropyl-β-cyclodextrin |
| IV | intravenously |
| IP | intraperitoneally |
| NHL | Non-Hodgkin Lymphoma |
| aNHL | aggressive Non-Hodgkin Lymphoma |
| iNHL | indolent Non-Hodgkin Lymphoma |
| DLBCL | Diffuse Large B-Cell Lymphomas |

Unless defined otherwise, all technical and scientific terms used herein have the same meaning as is commonly understood by one of skill in the art to which this disclosure belongs. All patents and publications referred to herein are incorporated by reference in their entirety.

As used herein, the term “cancer” refers to a cellular disorder characterized by uncontrolled or dysregulated cell proliferation, decreased cellular differentiation, inappropriate ability to invade surrounding tissue, and/or ability to establish new growth at ectopic sites. The term “cancer” includes solid tumors and non-solid tumors, such as, for example, hematological tumors. The term “cancer” encompasses diseases of skin, tissues, organs, bone, cartilage, blood, and vessels. The term “cancer” further encompasses primary and metastatic cancers.

As used herein, the term “autoimmune disease” refers to a disorder arising from an abnormal immune response to a normal body part. The term “autoimmune disease” encompasses disorders including, but not limited to, Rheumatoid Arthritis (RA), Granulomatosis with Polyangiitis (GPA) (Wegener’s Granulomatosis), and Microscopic Polyangiitis (MPA).

The term “CD38” refers to any native CD38, unless otherwise indicated. The term “CD38” encompasses “full-length,” unprocessed CD38 as well as any form of CD38 that results from processing within the cell. The term also encompasses naturally occurring variants of CD38, e.g., splice variants, allelic variants, and isoforms.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

A “blocking” antibody or an “antagonist” antibody is one which inhibits or reduces biological activity of the antigen it binds, such as, e.g., CD38. In a certain embodiment, blocking antibodies or antagonist antibodies substantially or completely inhibit the biological activity of the antigen. Desirably, the biological activity is reduced by 10%, 20%, 30%, 50%, 70%, 80%, 90%, 95%, or even 100%.

The term “anti-CD38 antibody” or “an antibody that binds to CD38” refers to an antibody that is capable of binding CD38 with sufficient affinity such that the antibody is useful as a diagnostic and/or therapeutic agent in targeting CD38. The extent of binding of an anti-CD38 antibody to an unrelated, non-CD38 protein is less than about 10% of the binding of the antibody to CD38 as measured, e.g., by a radioimmunoassay (RIA). In certain embodiments, an antibody that binds to CD38 has a dissociation constant (Kd) of ≤1 µM, ≤100 nM, ≤10 nM, ≤1 nM, or ≤0.1 nM.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

As used herein, the term “epitope” means a portion of an antigen to which an antibody specifically binds. Epitopes usually consist of chemically active (such as polar, nonpolar or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

As used herein, the term “effective amount” or “therapeutically effective amount” refers to an amount of a compound, or combination of one or more compounds that, when administered (either sequentially or simultaneously) elicits the desired biological or medicinal response, e.g., either destroys the target cancer cells or slows or arrests the progression of the cancer in a patient. The therapeutically effective amount may vary depending upon the intended application (in vitro or in vivo), or the patient and disease condition being treated, e.g., the weight and age of the patient, the severity of the disease condition, the manner of administration and the like, which may readily be determined by one skilled in the art. The term also applies to a dose that will induce a particular response in target cells, e.g., reduction of platelet adhesion and/or cell migration. For example, in some embodiments, the “therapeutically effective amount” as used herein refers to the amount of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and/or the amount of an anti-CD38 antibody that, when administered separately or in combination, have a beneficial effect. Further, it will be recognized by one skilled in the art that in the case of combination therapy, the amount of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof and/or the amount of the anti-CD38 antibody may be used in a “sub-therapeutic amount”, i.e., less than the therapeutically effective amount of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, or the anti-CD38 antibody alone.

The term “about” refers to approximately, in the region of, roughly, or around. When the term “about” is used in conjunction with a number or a numerical range, it means that the number or numerical range referred to is an approximation within experimental variability (or within statistical experimental error), and thus the number or numerical range may vary from, for example, between 1% and 15% of the stated number or numerical range. In general, the term “about” is used herein to modify a numerical value above and below the stated value by a variance of ±10%.

As used herein, “patient” generally means a mammal (e.g., human) who has been diagnosed with, exhibits symptoms of, or is otherwise believed to be afflicted with a disease, disorder, or condition (such as cancer).

The term “combination administration,” “administered in combination,” and “administering a combination” refers to administering of more than one pharmaceutically active ingredients (including, but not limited to, [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and an anti-CD38 antibody as disclosed herein) to a patient. Combination administration may refer to simultaneous administration or may refer to sequential administration of the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and an anti-CD38 antibody as disclosed herein.

The terms “simultaneous” and “simultaneously” refer to the administration of the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and an anti-CD38 antibody as disclosed herein, to a patient at the same time, or at two different time points that are separated by no more than 2 hours. The simultaneous administration of the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound 1-263a) or a pharmaceutically acceptable salt thereof, and an anti-CD38 antibody may be in a single dosage form or in separate dosage forms.

The terms “sequential” and “sequentially” refer to the administration of the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and an anti-CD38 antibody as disclosed herein, to a patient at two different time points that are separated by more than 2 hours, e.g., about 3 hours, about 4 hours, about 5 hours, about 8 hours, about 12 hours, 1 day, 2 days, 3 days, 4 days, 5 days, 6 days, 7 days or even longer.

The term “intermission” refers to a period that is subsequent to the administration of one or more particular pharmaceutically active ingredients to a patient in an intermittent regimen. Intermission refers to a rest period wherein a particular pharmaceutically active ingredient is not administered for at least one day.

The term “synergistic effect” refers to a situation where the combination of two or more agents produces a greater effect than the sum of the effects of each of the individual agents. The term encompasses not only a reduction in symptoms of the disorder to be treated, but also an improved side effect profile, improved tolerability, improved patient compliance, improved efficacy, or any other improved clinical outcome.

As used herein, the illustrative terms “include”, “such as”, “for example” and the like (and variations thereof, e.g., “includes” and “including”, “examples”), unless otherwise specified, are intended to be non-limiting. That is, unless explicitly stated otherwise, such terms are intended to imply “but not limited to”, e.g., “including” means including but not limited to.

Unless otherwise stated, structures depicted herein are meant to include chemical entities which differ only in the presence of one or more isotopically enriched atoms. For example, chemical entities having the present structure except for the replacement of a hydrogen atom by a deuterium or tritium, or the replacement of a carbon atom by a ¹³C- or ¹⁴C-enriched carbon are within the scope of the invention.

Unless stereochemical configuration is denoted, structures depicted herein are meant to include all stereochemical forms of the structure, i.e., the R and S configurations for each asymmetric center. Therefore, unless otherwise indicated, single stereochemical isomers as well as enantiomeric, racemic and diastereomeric mixtures of the present chemical entities are within the scope of the invention. When a stereochemical configuration is denoted for a compound, the diastereoisomeric or enantiomeric excess of the compound is at least 99.0%, 99.5%, 99.6%, 99.7%, 99.8% or 99.9%.

### SAE Inhibitor

The present disclosure provides a combination treatment for patients with cancer or autoimmune disease. The combination treatment includes, inter alia, administering to a subject in need thereof a therapeutically effective amount of at least one SAE inhibitor.

In some embodiments, the SAE inhibitor is [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate, or a pharmaceutically acceptable salt thereof, having the following structure:

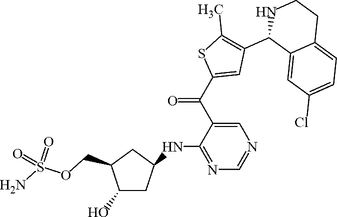 [SMILES: Cc1sc(C(=O)c2cncnc2N[C@@H]2C[C@H](COS(N)(=O)=O)[C@@H](O)C2)cc1[C@@H]1NCCc2ccc(Cl)cc21]

[(1R,2S,4R)-4-{[5-(4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate is also referred to herein as Compound I-263a.

In some embodiments, the SAE inhibitor is [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate, or a pharmaceutically acceptable salt thereof.

In some embodiments, the SAE inhibitor is [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate, or Compound I-263a.

SAE inhibitors, as disclosed herein, are described, for example, in US 2016/0009744 and US 9,695,154. They may be prepared by methods known to one skilled in the art and/or according to the methods described in US 2016/0009744 and US 9,695,154, which is hereby incorporated by reference in its entirety. Central to the mechanism of action of useful SAE inhibitors, such as Compound I-263a, in combinations and methods of the present disclosure is production of type 1 IFNs and induction of an innate immune response with activation of both natural killer (NK) cells and macrophages. Biochemical assays have demonstrated that Compound I-263a is a mechanism-based inhibitor of SUMO-activating enzyme that potently inhibits enzyme activity by forming a covalent adduct with SUMO. Strong selectivity for SUMO-activating enzyme was observed over the other closely related ubiquitin-activating enzymes ubiquitin-activating enzyme, Nedd8-activating enzyme, and autophagy related 7 enzyme. Selective and potent inhibition of SUMO-activating enzyme and SUMOylation by Compound I-263a has been demonstrated in cultured mouse and human tumor cell lines and the antiproliferative activity of Compound I-263a has been determined in a panel of 7 mouse hematologic and solid tumor cell lines.

In some embodiments, the SAE inhibitor is [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a crystalline form thereof.

In some embodiments, the SAE inhibitor or a pharmaceutical salt thereof is crystalline form 1 of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a), as described in U.S. published application number US 2016/0009744.

In some embodiments, the SAE inhibitor or a pharmaceutical salt thereof is crystalline form 2 of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a), as described in U.S. published application number US 2016/0009744.

In some embodiments, the SAE inhibitor or a pharmaceutical salt thereof is crystalline form 3 of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a), described in U.S. published application number US 2016/0009744.

### Anti-CD38 Antibodies

The present disclosure provides a combination treatment that includes, inter alia, administering to a subject in need thereof a therapeutically effective amount of at least one anti-CD38 antibody (e.g., daratumumab).

CD38 is a type II transmembrane glycoprotein expressed on hemopoietic cells such as medullary thymocytes, activated T- and B-cells, resting NK cells and monocytes, lymphnode germinal center lymphoblasts, plasma B cells, intra-follicular cells and dendritic cells. A portion of normal bone marrow cells, particular precursor cells as well as unbilical cord cells are CD38-positive. In addition to lymphoid precursor cells, CD38 is expressed on erythrocytes and on platelets, and expression is also found in some solid tissues Such as gut, brain, prostate, bone, and pancreas. Mature resting T- and B-cells express limited to no surface CD38.

Any anti-CD38 antibody may be used in the methods described in the present disclosure, provided that the anti-CD38 antibody induces in vitro killing of CD38-expressing cells by antibody-dependent cell-mediated cytotoxicity (ADCC), antibody-dependent cellular phagocytosis (ADCP), complement dependent cytotoxicity (CDC), apoptosis, or modulation of CD38 enzymatic activity. The variable regions of the anti-CD38 antibodies may be obtained from existing anti-CD38 antibodies, and cloned as full length antibodies using standard methods. Exemplary variable regions binding CD38 that may be used are described, e.g., in Intl. Pat. Publ. Nos. WO05/103083, WO06/125640, WO07/042309, WO08/047242, WO12/092612, WO06/099875 and WO11/154453A1.

In some embodiments, the anti-CD38 antibody is selected from the group consisting of mezagitamab, daratumumab and isatuximab. In some embodiments, the anti-CD38 antibody is mezagitamab. In some embodiments, the anti-CD38 antibody is daratumumab. In some embodiments, the anti-CD38 antibody is isatuximab. In some embodiments, the anti-CD38 antibody is daratumumab formulated with a hyaluronidase, such as hyaluronidase-fihj.

Mezagitamab is a human monoclonal antibody that binds specifically to the CD38 transmembrane glycoprotein expressed on the surface of cells. Mezagitamab can induce apoptosis of cells which express a high density of the target antigen and/or recruit effectors of the immune system to lyse bound cells. Mezagitamab is described in U.S. Pat. No. 8,362,211.

Daratumumab is an immunoglobulin G1 kappa (IgG1κ) human monoclonal antibody against CD38 antigen, produced in a mammalian cell line (Chinese Hamster Ovary [CHO]) using recombinant DNA technology. Daratumumab was initially approved by the FDA in 2015 for treating multiple myeloma. In some embodiments, daratumumab is commercially available as Darzalex®. In some embodiments, daratumumab is a biosimilar or an interchangeable product. In some embodiments, daratumumab is formulated with a hyaluronidase, such as hyaluronidase-fihj. In some embodiments, daratumumab is commercially available as Darzalex Faspro®. In one embodiment, the dosage of DARZALEX FASPRO is (1,800 mg daratumumab and 30,000 units hyaluronidase) administered subcutaneously into the abdomen over approximately 3 to 5 minutes.

Additional anti-CD38 antibodies used in the methods described in the present disclosure include but are not limited to: MOR03087 (also known as MOR202) described in U.S. Pat. No. 8,088,896; SG303 described in Yu et al., BMC Biotechnology, 19:28, 2019; mAb024 and mAb003 described in U.S. Pat. No. 7,829,693; and the anti-CD38 antibodies described in U.S. Pat. No. 9,603,927.

Additional anti-CD38 antibodies used in the methods described in the present disclosure may also be selected de novo from, e.g., a phage display library, where the phage is engineered to express human immunoglobulins or portions thereof. Such as Fabs, single chain antibodies (scFV), or unpaired or paired antibody variable regions (Knappik et al., J Mol Biol 296: 57-86, 2000; Krebs et al., J Immunol Meth 254:67-84, 2001; Vaughan et al., Nature Biotechnology 14:309-314, 1996; Sheets et al., PITAS (USA) 95:6157-6162, 1998: Hoogenboom and Winter, J Mol Biol 227:381, 1991; Marks et al., J Mol Biol 222:581, 1991). CD38 binding variable domains may be isolated from e.g., phage display libraries expressing antibody heavy and light chain variable regions as fusion proteins with bacteriophage pX coat protein as described in Shi et al., J. Mol. Biol. 397:385-96, 2010 and PCT Intl. Publ. No. WO09/085462). The antibody libraries can be screened for binding to human CD38 extracellular domain, obtained positive clones further characterized, Fabs isolated from the clone lysates, and subsequently cloned as full length antibodies. Such phage display methods for isolating human antibodies are established in the art. See for example: U.S. Pat. No. 5,223,409; U.S. Pat. No. 5,403,484; and U.S. Pat. No. 5,571,698, U.S. Pat. No. 5,427,908, U.S. Pat. No. 5,580,717, U.S. Pat. No. 5,969,108, U.S. Pat. No. 6,172,197, U.S. Pat. No. 5,885,793; U.S. Pat. No. 6,521,404: U.S. Pat. No. 6,544,731; U.S. Pat. No. 6,555,313; U.S. Pat. No. 6,582,915; and U.S. Pat. No. 6,593,081.

In some embodiments, the anti-CD38 antibody used in the methods (and kits) described herein is daratumumab or an anti-CD38 antibody that binds to the same epitope as daratumumab. In some embodiments, the anti-CD38 antibody is daratumumab. In some embodiments, the daratumumab is administered in an injectable formulation that comprises daratumumab and hyaluronidase. In some embodiments, the hyaluronidase is hyaluronidase-fihj.

In some embodiments, the anti-CD38 antibody used in the methods (and kits) described herein is mezagitamab or an anti-CD38 antibody that binds to the same epitope as mezagitamab. In some embodiments, the anti-CD38 antibody is mezagitamab.

### Methods of Treating Cancer or Autoimmune Disease

In some embodiments, the present disclosure relates to a method of treating a disorder which is cancer or autoimmune disease in a patient by administering to a patient in need of said treating a combination of an SAE inhibitor or pharmaceutically acceptable salt thereof and one or more anti-CD38 antibodies.

In some embodiments, the present disclosure relates to a method of treating a disorder which is cancer or autoimmune disease by administering to a patient in need of said treating a combination of an SAE inhibitor and an anti-CD38 antibody.

In some embodiments, the present disclosure relates to the use of an SAE inhibitor in combination with an anti-CD38 antibody for the treatment of a disorder which is cancer or autoimmune disease in a patient.

In some embodiments, the present disclosure relates to a composition comprising an SAE inhibitor for use in treating a disorder which is cancer or autoimmune disease in a patient, wherein the patient is also treated with an anti-CD38 antibody. In some aspects, the disclosure relates to a composition comprising an SAE inhibitor for use in treating a disorder which is cancer or autoimmune disease in a patient, wherein the SAE inhibitor is in combination with the anti-CD38 antibody. In some embodiments, the SAE inhibitor can be administered simultaneously or sequentially with the anti-CD38 antibody.

In some embodiments, the present disclosure relates to methods of treating a disorder which is cancer or autoimmune disease comprising administering to a patient in need of such treatment, a therapeutically effective amount of a combination of an SAE inhibitor and an anti-CD38 antibody.

In some embodiments, the present disclosure relates to a method of treating a disorder which is cancer or autoimmune disease by administering to a patient a combination of Compound I-263a, or pharmaceutically acceptable salt thereof, and an anti-CD38 antibody.

In another aspect, the present disclosure relates to the use of Compound I-263a, or a pharmaceutically acceptable salt thereof, in combination with an anti-CD38 antibody for the treatment of a disorder which is cancer or autoimmune disease.

In some embodiments, the methods of treating a disorder which is cancer or autoimmune disease, as described herein, can include a combination of an SAE inhibitor, an anti-CD38 antibody, and one or more additional therapeutic agents. In some embodiments, the one or more additional therapeutic agents can be chemotherapeutic agents. In some embodiments, the one or more additional therapeutic agents can include, but are not limited to, lenalidomide, dexamethasone, bortezomib, melphalan, prednisone, pomalidomide, fludarabine, cyclophosphamide, doxorubicin, vincristine, methotrexate anthracycline-based chemotherapeutic agents, , methylprednisolone, glucocorticoids, Ibritumomab tiuxetan, acetaminophen, antihistamines, and combinations thereof. In another embodiment, the anti-CD38 antibody is coadministered with lenalidomide, dexamethasone, bortezomib, melphalan, prednisone, pomalidomide, and combinations thereof. In some embodiments, the anti-CD38 antibody is coadministered with lenalidomide and dexamethasone. In some embodiments, the anti-CD38 antibody is coadministered with bortezomib. In some embodiments, the anti-CD38 antibody is coadministered with melphalan and prednisone. In some embodiments, the anti-CD38 antibody is coadministered with pomalidomide and dexamethasone.

In some embodiments, the disorder is cancer.

In some embodiments, the cancer is a solid tumor. Non-limiting examples of solid tumors include pancreatic cancer; bladder cancer, including invasive bladder cancer; colorectal cancer; thyroid cancer; gastric cancer; breast cancer, including metastatic breast cancer; prostate cancer, including androgen-dependent and androgen-independent prostate cancer; renal cancer, including, e.g., metastatic renal cell carcinoma; liver cancer including e.g. hepatocellular carcinoma and intrahepatic bile duct cancer; lung and bronchus cancer including non-small cell lung cancer (NSCLC), squamous lung cancer, brochioloalveolar carcinoma (BAC), adenocarcinoma of the lung, and small cell lung cancer (SCLC); ovarian cancer including, e.g., progressive epithelial and primary peritoneal cancer; cervical cancer; uterine cancer including e.g. uterine corpus and uterine cervix; endometrial cancer; esophageal cancer; head and neck cancer, including, e.g., squamous cell carcinoma of the head and neck, nasopharyngeal caner, oral cavity and pharynx; melanoma; neuroendocrine cancer, including metastatic neuroendocrine tumors; brain cancer, including, e.g., glioma/glioblastoma, anaplastic oligodendroglioma, adult glioblastoma multiforme, adult anaplastic astrocytoma, and medulloblastoma; neuroendocrine cancer, including metastatic neuroendocrine tumors; bone cancer; gastro-esophageal junction cancer, and soft tissue sarcoma.

In some embodiments, the cancer is a hematological cancer. Non-limiting examples of hematologic malignancies include acute myeloid leukemia (AML); chronic myelogenous leukemia (CML), including accelerated CML and CML blast phase (CML-BP); acute lymphoblastic leukemia (ALL); chronic lymphocytic leukemia (CLL); Hodgkin’s lymphoma (HL); non-Hodgkin’s lymphoma (NHL), including B-cell lymphoma, T-cell lymphoma, follicular lymphoma (FL), marginal zone lymphoma (MZL), mantle cell lymphoma (MCL), diffuse large B-cell lymphoma (DLBCL), and Burkitt lymphoma; multiple myeloma (MM); amyloidosis; Waldenstrom’s macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes. In some embodiments, the cancer is chronic lymphocytic leukemia (CLL), Hodgkin’s lymphoma, or non-Hodgkin’s lymphoma including follicular lymphoma (FL), marginal zone lymphoma (MZL), mantle cell lymphoma (MCL), Diffuse large B-cell lymphoma (DLBCL) and Burkitt lymphoma.

In some embodiments, the cancer is multiple myeloma (MM). In some embodiments, the cancer is relapsed and/or refractory multiple myeloma (RRMM).

In some embodiments, the disorder is a CD38-positive cancer.

In some embodiments, the cancer is a CD38-positive solid tumor. Non-limiting examples of solid tumors include pancreatic cancer; bladder cancer, including invasive bladder cancer; colorectal cancer; thyroid cancer; gastric cancer; breast cancer, including metastatic breast cancer; prostate cancer, including androgen-dependent and androgen-independent prostate cancer; renal cancer, including, e.g., metastatic renal cell carcinoma; liver cancer including e.g. hepatocellular carcinoma and intrahepatic bile duct cancer; lung and bronchus cancer including non-small cell lung cancer (NSCLC), squamous lung cancer, brochioloalveolar carcinoma (BAC), adenocarcinoma of the lung, and small cell lung cancer (SCLC); ovarian cancer including, e.g., progressive epithelial and primary peritoneal cancer; cervical cancer; uterine cancer including e.g. uterine corpus and uterine cervix; endometrial cancer; esophageal cancer; head and neck cancer, including, e.g., squamous cell carcinoma of the head and neck, nasopharyngeal caner, oral cavity and pharynx; melanoma; neuroendocrine cancer, including metastatic neuroendocrine tumors; brain cancer, including, e.g., glioma/glioblastoma, anaplastic oligodendroglioma, adult glioblastoma multiforme, adult anaplastic astrocytoma, and medulloblastoma; neuroendocrine cancer, including metastatic neuroendocrine tumors; bone cancer; gastro-esophageal junction cancer, and soft tissue sarcoma.

In some embodiments, the cancer is CD38-positive hematological malignancy. CD3 8-positive hematological malignancy refers to a hematological malignancy characterized by the presence of tumor cells expressing CD38 including leukemias, lymphomas and myeloma. Examples of such CD38-positive hematological malignancies include precursor B-cell lymphoblastic leukemia/lymphoma and B-cell non-Hodgkin’s lymphoma; acute promyelocytic leukemia, acute lymphoblastic leukemia and mature B-cell neoplasms, such as B-cell chronic lymphocytic leukemia (CLL)/small lymphocytic lymphoma (SLL), B-cell acute lymphocytic leukemia, B-cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B-cell lymphoma (MALT type, nodal and splenic type), hairy cell leukemia, diffuse large B-cell lymphoma (DLBCL), Burkitt’s lymphoma (BL), plasmacytoma, multiple myeloma, plasma cell leukemia, post-transplant lymphoproliferative disorder, Waldenstrom’s macroglobulinemia, plasma cell leukemias and anaplastic large-cell lymphoma (ALCL).

In one embodiment of the invention disclosed herein, the CD-38 positive hematological malignancy is multiple myeloma. In one embodiment of the invention disclosed herein, the CD-38 positive hematological malignancy is relapsed and/or refractory multiple myeloma.

In one embodiment of the invention disclosed herein, the CD-38 positive hematological malignancy is diffuse large B-cell lymphoma (DLBCL).

In one embodiment of the invention disclosed hereinthe CD-38 positive hematological malignancy is follicular lymphoma (FL).

In one embodiment of the invention disclosed herein, the CD-38 positive hematological malignancy is mantle cell lymphoma (MCL).

In one embodiment of the invention disclosed herein, the CD38-positive hematological malignancy is multiple myeloma, acute lymphoblastic leukemia (ALL), non-Hodgkin’s lymphoma, diffuse large B-cell lymphoma (DLBCL), Burkitt’s lymphoma (BL), follicular lymphoma (FL) or mantle-cell lymphoma (MCL).In one embodiment of the present invention, the disorder involving cells expressing CD38 is Hodgkin’s lymphoma.

Other examples of disorders involving cells expressing CD38 include malignancies derived from T and NK cells including: mature T cell and NK cell neoplasms including T-cell prolymphocytic leukemia, T-cell large granular lymphocytic leukemia, aggressive NK cell leukemia, adult T-cell leukemia/lymphoma, extranodal NK/T cell lymphoma, nasal type, 78 enteropathy-type T-cell lymphoma, hepatosplenic T-cell lymphoma, subcutaneous panniculitis-like T-cell lymphoma, blastic NK cell lymphoma, Mycosis Fungoides/Sezary Syndrome, primary cutaneous CD30 positive T-cell lymphoproliferative disorders (primary cutaneous anaplastic large cell lymphoma C-ALCL, lymphomatoid papulosis, borderline lesions), angioimmunoblastic T-cell lymphoma, peripheral T-cell lymphoma unspecified, and anaplastic large cell lymphoma.

Examples of malignancies derived from myeloid cells include acute myeloid leukemia, including acute promyelocytic leukemia, and chronic myeloproliferative diseases, including chronic myeloid leukemia.

In some embodiments, the cancer is relapsed. In some embodiments, relapsed cancer is cancer which has returned after a period of time in which no cancer could be detected.

In some embodiments, the cancer is refractory. In some embodiments, refractory cancer does not respond to cancer treatment; it is also known as resistant cancer. In some embodiments, the cancer is resistant to daratumumab. In some embodiments, the cancer does not respond to the treatment of daratumumab. In some embodiments, the cancer is daratumumab-resistant recurrent cancer. In some embodiments, the patient has become refractory to a daratumumab-containing regimen. In some embodiments, the tumor is unresectable. In some embodiments, an unresectable tumor is unable to be removed by surgery. In some embodiments, the cancer has not been previously treated. In some embodiments, the cancer is locally advanced. In some embodiments, “locally advanced” refers to cancer that is somewhat extensive but still confined to one area. In some instances, “locally advanced” may refer to a small tumor that hasn’t spread but has invaded nearby organs or tissues that make it difficult to remove with surgery alone. In some embodiments, the cancer is metastatic. In some embodiments, metastatic cancer is a cancer that has spread from the part of the body where it started (the primary site) to other parts of the body.

In some embodiments, the patient has relapsed or refractory CD38-positive multiple myeloma. In some embodiments, the patient has both CD38-positive multiple myeloma and relapsed or refractory multiple myeloma. In some embodiments, the patient has relapsed or refractory CD38-positive non-Hodgkin lymphoma. In some embodiments, the patient has both CD38-positive non-Hodgkin lymphoma and relapsed or refractory non-Hodgkin lymphoma.

In some embodiments, the patient has relapsed or refractory CD38-positive aggressive non-Hodgkin lymphoma. In some embodiments, the patient has relapsed or refractory CD38-positive aggressive non-Hodgkin lymphoma and has progressed on at least one prior treatment regimen.

In some embodiments, the patient has relapsed or refractory CD38-positive indolent non-Hodgkin lymphoma. In some embodiments, the patient has relapsed or refractory CD38-positive indolent non-Hodgkin lymphoma and has progressed on at least two prior treatment regimens. In some embodiments, the patient has relapsed or refractory CD38-positive indolent non-Hodgkin lymphoma and is refractory to any anti-CD38 monoclonal antibody. In some embodiments, the patient has relapsed or refractory CD38-positive indolent non-Hodgkin lymphoma and has progressed on at least two prior treatment regimens and is refractory to any anti-CD38 monoclonal antibody.

In some embodiments, the disorder is an SAE-mediated disorder other than cancer. In some embodiments, the disorder is an autoimmune disease.

### Medicament

In some embodiments, the present disclosure relates to a medicament for use in treating a disorder which is cancer or autoimmune disease in a patient in need of such treatment. The medicament comprises an SAE inhibitor and an anti-CD38 antibody, and is in single dosage form or in separate dosage forms.

In some embodiments, the medicaments, as described herein, can include a combination of an SAE inhibitor, an anti-CD38 antibody, and optionally one or more additional therapeutic agents.

In some embodiments, the present disclosure relates to the use of an SAE inhibitor in the manufacture of a medicament for treating a disorder which is cancer or autoimmune disease, wherein the SAE inhibitor is administered with an anti-CD38 antibody, and wherein the medicament is in single dosage form or in separate dosage forms. In some embodiments, the SAE inhibitor is administered with an anti-CD38 antibody and one or more additional therapeutic agents.

In some embodiments, the present disclosure relates to the use of an SAE inhibitor for the manufacture of a medicament in treating a disorder which is cancer or autoimmune disease in a patient, wherein the patient is also treated with an anti-CD38 antibody, and optionally one or more additional therapeutic agents. In some embodiments, the SAE inhibitor may be administered simultaneously or sequentially with the anti-CD38 antibody. In some aspects, the present disclosure relates to the use of an SAE inhibitor for the manufacture of a medicament in treating a disorder which is cancer or autoimmune disease in a patient, wherein the SAE inhibitor is in combination with an anti-CD38 antibody, and optionally one or more additional therapeutic agents. In some embodiments, the SAE inhibitor is in the same composition as the anti-CD38 antibody. In some embodiments, the SAE inhibitor is in a separate composition as the anti-CD38 antibody. In some embodiments, the SAE inhibitor is in the same composition as one or more additional therapeutic agents. In some embodiments, the SAE inhibitor is in the same composition as the anti-CD38 antibody, and optionally one or more additional therapeutic agents. In some embodiments, the SAE inhibitor is in a separate composition as one or more additional therapeutic agents. In some embodiments, the SAE inhibitor is in a separate composition as the anti-CD38 antibody, and optionally one or more additional therapeutic agents.

In another aspect, the present disclosure relates to the use of Compound I-263a, or a pharmaceutically acceptable salt thereof in combination with an anti-CD38 antibody in the manufacture of a medicament for use in treating a disorder which is cancer or autoimmune disease. In some embodiments, the present disclosure relates to the use of Compound I-263a, or a pharmaceutically acceptable salt thereof in combination with an anti-CD38 antibody, and optionally one or more additional therapeutic agents in the manufacture of a medicament for use in treating a disorder which is cancer or autoimmune disease.

In another aspect, the present disclosure relates to the use of Compound I-263a, or a pharmaceutically acceptable salt thereof, in the manufacture of a medicament for treating a disorder which is cancer or autoimmune disease, wherein Compound I-263a or a pharmaceutically acceptable salt thereof is administered with an anti-CD38 antibody, and optionally one or more additional therapeutic agents.

In some embodiments, the one or more additional therapeutic agents can be chemotherapeutic agents. In some embodiments, the one or more additional therapeutic agents can include, but are not limited to, fludarabine, cyclophosphamide, doxorubicin, vincristine, methotrexate anthracycline-based chemotherapeutic agents, prednisone, methylprednisolone, glucocorticoids, Ibritumomab tiuxetan, acetaminophen, antihistamines, and combinations thereof. In some embodiments, the anti-CD38 antibody is coadministered with lenalidomide, dexamethasone, bortezomib, melphalan, prednisone, pomalidomide, and combinations thereof. In some embodiments, the anti-CD38 antibody is coadministered with lenalidomide and dexamethasone. In some embodiments, the anti-CD38 antibody is coadministered with bortezomib. In some embodiments, the anti-CD38 antibody is coadministered with melphalan and prednisone. In some embodiments, the anti-CD38 antibody is coadministered with pomalidomide and dexamethasone.

### Administration of the Combination

Compound I-263a or a pharmaceutically acceptable salt thereof, may be administered in combination with the anti-CD38 antibody, and optionally one or more additional therapeutic agents, in a single dosage form or as a separate dosage forms. In some embodiments, when administered as a separate dosage form, the anti-CD38 antibody may be administered prior to, at the same time as, or following administration of 1-263a or a pharmaceutically acceptable salt thereof. In some embodiments, when administered as a separate dosage form, one or more doses of I-263a or a pharmaceutically acceptable salt thereof, may be administered prior to the anti-CD38 antibody. In some embodiments, the anti-CD38 antibody is administered prior to the administration of Compound I-263a or a pharmaceutically acceptable salt thereof. As used herein, the administration in “combination” of Compound I-263a or a pharmaceutically acceptable salt thereof, an anti-CD38 antibody, and optionally one or more additional therapeutic agents refers not only to simultaneous or sequential administration of the agents, but also to the administration of the agents during a single treatment cycle, as understood by one skilled in the art. When Compound I-263a or a pharmaceutically acceptable salt thereof is administered in combination with the anti-CD38 antibody, and optionally one or more additional therapeutic agents, a therapeutically effective amount of the combination is administered.

The SAE inhibitor may be administered by any method known to one skilled in the art. For example, in some embodiments, the SAE inhibitor may be administered in the form of a pharmaceutical composition of the SAE inhibitor and a pharmaceutically acceptable carrier, such as those described herein. In some embodiments, the pharmaceutical composition is suitable for oral administration. In some embodiments, the pharmaceutical composition is a tablet or a capsule that is suitable for oral administration. In some other embodiments, the pharmaceutical composition is a liquid dosage form suitable for oral administration. In some embodiments, the pharmaceutical composition is suitable for intravenous administration. In some embodiments, the pharmaceutical composition is suitable for subcutaneous administration. In some embodiments, these compositions optionally further comprise one or more additional therapeutic agents.

In some embodiments, the SAE inhibitor is administered by intravenous infusion. In some embodiments, the SAE inhibitor is administered as a 60 ±10 minute IV infusion.

The anti-CD38 antibody may be administered by any method known to one skilled in the art. In some embodiments, the anti-CD38 antibody is administered intravenously (IV). In some embodiments, the anti-CD38 antibody is administered subcutaneously (SC). In some embodiments, the anti-CD38 antibody is administered orally. For example, the anti-CD38 antibody may be administered in the form of a second composition, in some embodiments, a pharmaceutical composition of the anti-CD38 antibody and a pharmaceutically acceptable carrier, such as those described herein. In some aspects, the pharmaceutical composition is suitable for oral administration. In some embodiments, the pharmaceutical composition is a tablet or a capsule that is suitable for oral administration. In some other embodiments, the pharmaceutical composition is a liquid dosage form suitable for oral administration. In some embodiments, these compositions optionally further comprise one or more additional therapeutic agents.

In some embodiments, the anti-CD38 antibody is administered by subcutaneous injection.

The amounts or suitable doses of the methods of this disclosure depends upon a number of factors, including the nature of the severity of the condition to be treated, the particular inhibitor, the route of administration and the age, weight, general health, and response of the individual patient. In some embodiments, the suitable dose level is one that achieves a therapeutic response as measured by tumor regression, or other standard measures of disease progression, progression free survival or overall survival. In some embodiments, the suitable dose level is one that achieves this therapeutic response and also minimizes any side effects associated with the administration of the therapeutic agent. The suitable dose levels may be ones that prolong the therapeutic response and/or prolong life.

It will be understood that a suitable dose of the SAE inhibitor, the anti-CD38 antibody, and optionally one or more additional therapeutic agents may be taken at any time of the day or night. In some embodiments, a suitable dose of each agent is taken in the morning. In some other embodiments, a suitable dose of each agent is taken in the evening. In some embodiments, a suitable dose of each of the agents is taken both in the morning and the evening. It will be understood that a suitable dose of each agent may be taken with or without food. In some embodiments a suitable dose of an agent is taken with a meal. In some embodiments a suitable dose of an agent is taken while fasting.

In some embodiments, Compound I-263a or a pharmaceutically acceptable salt thereof is administered on a daily schedule. In some embodiments, Compound I-263a or a pharmaceutically acceptable salt thereof is administered every other day. In some embodiments, Compound I-263a or a pharmaceutically acceptable salt thereof is administered once every three days. In some embodiments, Compound I-263a or a pharmaceutically acceptable salt thereof is administered on a twice-weekly schedule. In some embodiments, Compound I-263a or a pharmaceutically acceptable salt thereof is administered on a three times a week schedule. In some embodiments, Compound I-263a or a pharmaceutically acceptable salt thereof is administered on a weekly schedule. In some embodiments, Compound I-263a or a pharmaceutically acceptable salt thereof is administered on a once every two weeks schedule. In some embodiments, Compound I-263a or a pharmaceutically acceptable salt thereof is administered on a once every month schedule.

In some embodiments, Compound I-263a or a pharmaceutically acceptable salt thereof is administered at least 3 times on alternate days within a 7-day cycle. In some embodiments, Compound I-263a or a pharmaceutically acceptable salt thereof is administered on day 1 and day 4 of a 7-day cycle. In some embodiments, Compound I-263a or a pharmaceutically acceptable salt thereof is administered on consecutive days in a 7-day cycle followed by an intermission. In some embodiments, Compound I-263a or a pharmaceutically acceptable salt thereof is administered for 2 consecutive days followed by an intermission of 5 consecutive days for at least one 7-day cycle. In some embodiments, Compound I-263a or a pharmaceutically acceptable salt thereof is administered for 3 consecutive days followed by an intermission of 4 consecutive days for at least one 7-day cycle. In some embodiments, Compound I-263a or a pharmaceutically acceptable salt thereof is administered for 4 consecutive days followed by an intermission of 3 consecutive days for at least one 7-day cycle. In some embodiments, Compound I-263a or a pharmaceutically acceptable salt thereof is administered for 5 consecutive days followed by an intermission of 2 consecutive days for at least one 7-day cycle. In some embodiments, there will be periods of rest between one or more of the 7-day treatment cycles. In some embodiments, there will be a 7-day rest between one or more of the 7-day treatment cycles.

The present description contemplates administration of the drug for one or more treatment cycles, for example, 1, 2, 3, 4, 5, 6, or more, treatment cycles. In some embodiments, a treatment cycle is about 7 days to about 56 days, or more. In some embodiments, a treatment cycle is 7 days, 14 days, 21 days, 28 days, 35 days, 42 days, 49 days, or 56 days. In some embodiments, a treatment cycle is 14 days, 21 days, 28 days, or 35 days. In some embodiments, there will be periods of rest within or between one or more of the treatment cycles. For example, in some embodiments, there will be a period of rest at the end of the treatment cycle. In some embodiments, there will be a period of rest between the second and third treatment cycle but not the first and second treatment cycle. In another embodiment, there might be a period of rest between the first and second treatment cycle but not the second and third treatment cycle. Dosing schedules include, for example, administering the SAE inhibitor once during a treatment schedule, e.g., on day 1 of a 21 day cycle or on day 1 of a 28 day cycle, twice during a treatment cycle, e.g., on days 1 and 15 of a 21 day cycle or on days 1 and 15 of a 28 day cycle, three times during a treatment cycle, e.g., on days 1, 8 and 15 of a 21 day cycle or on days 1, 8 and 15 of a 28 day cycle, four times during a treatment cycle, e.g., on days 1, 4, 8, and 11 of a 14 days cycle, days 0, 3, 7, and 10 of a 14 day cycle, on days 1, 4, 8, and 11 of a 21 day cycle, on days 1, 8, 15, and 22 of a 28 day cycle, or on days 1, 4, 8, and 11 of a 28 day cycle, five times during a treatment cycle, e.g., on days 1, 4, 8, 11, and 15 of a 28 days cycle, six times during a treatment cycle, e.g., on days 1, 4, 8, 11, 15, and 18 of a 21 day cycle or on days 1, 4, 8, 11, 15, and 18 of a 28 day cycle, eight times during a treatment cycle, e.g. days 1, 4, 8, 11, 15, 18, 22 and 25 of a 28 day cycle or days 1, 4, 8, 11, 15, 18, 22 and 25 of a 28 day, ten times during a 35 day cycle, e.g. days 1, 4, 8, 11, 15, 18, 22, 25, 29 and 33 of a 35 day cycle. Day 0 of a treating cycle is one day immediately before the day that the treatment cycle starts. Other dosage schedules are encompassed by the present invention.

In some embodiments, Compound I-263a or a pharmaceutically acceptable salt thereof is administered within a 14-day cycle. In some embodiments, Compound I-263a or a pharmaceutically acceptable salt thereof is administered twice weekly on days on 0, 3, 7, and 10 of the 14 day cycle. In some embodiments, Compound I-263a or a pharmaceutically acceptable salt thereof is administered twice weekly on days on 1, 4, 8, and 11 of the 14 day cycle.

In some embodiments, Compound I-263a or a pharmaceutically acceptable salt thereof is administered within a 21 day cycle. In some embodiments, Compound I-263a or a pharmaceutically acceptable salt thereof is administered twice weekly on days on 1, 4, 8, 11, 15, and 18 of the 21 day cycle. In some embodiments, Compound I-263a or a pharmaceutically acceptable salt thereof is administered on days 1, 4, 8, and 11 of a 21 day cycle. In some embodiments, Compound I-263a or a pharmaceutically acceptable salt thereof is administered on days 1 and 8 of a 21 day cycle.

In some embodiments, Compound I-263a or a pharmaceutically acceptable salt thereof is administered within a 28 day cycle. In some embodiments, Compound I-263a or a pharmaceutically acceptable salt thereof is administered twice weekly on days on 1, 4, 8, 11, 15, and 18 of the 28 day cycle. In some embodiments, Compound I-263a or a pharmaceutically acceptable salt thereof is administered twice weekly on days on 1, 4, 8, 11, and 15 of the 28 day cycle.. In some embodiments, Compound I-263a or a pharmaceutically acceptable salt thereof is administered twice weekly on days on 1, 4, 8, 11, and 15 of the 28 day cycle in cycles 1 and 2 followed by every 2 weeks in cycles 3 through 6, then monthly. In some embodiments, Compound I-263a or a pharmaceutically acceptable salt thereof is administered weekly on days on 1, 8, 15, and 22 of the 28 day cycle. In some embodiments, Compound I-263a or a pharmaceutically acceptable salt thereof is administered once weekly on days on 1, 8, 15, and 22 of the 28 day cycle. In some embodiments, Compound I-263a or a pharmaceutically acceptable salt thereof is administered once weekly on days on 1, 8, 15, and 22 of the 28 day cycle in cycles 1 and 2 followed by every 2 weeks in cycles 3 through 6, then monthly. In some embodiments, Compound I-263a or a pharmaceutically acceptable salt thereof is administered once weekly on days on 1 and 15 of the 28 day cycle. In some embodiments, Compound I-263a or a pharmaceutically acceptable salt thereof is administered once on day 1 of the 28 day cycle.

In some embodiments, Compound I-263a or a pharmaceutically acceptable salt thereof is administered within a 35 day cycle. In some embodiments, Compound I-263a or a pharmaceutically acceptable salt thereof is administered twice weekly on days on 1, 4, 8, 11, 15, 18, 22, 25, 29 and 33 of the 35 day cycle.

In some embodiments, Compound I-263a or a pharmaceutically acceptable salt thereof is administered for a duration of 1 year or less. In some embodiments, Compound I-263a or a pharmaceutically acceptable salt thereof is administered for a duration of 1 year or more.

In some embodiments, the amount of Compound I-263a or a pharmaceutically acceptable salt thereof that is administered on each day of dosing is about 0.5 mg to about 300 mg. In some embodiments, the amount of Compound I-263a or a pharmaceutically acceptable salt thereof that is administered on each day of dosing is about 0.5 mg to about 250 mg. In some embodiments, the amount of Compound I-263a or a pharmaceutically acceptable salt thereof that is administered on each day of dosing is about 0.5 mg to about 200 mg. In some embodiments, the amount of Compound I-263a or a pharmaceutically acceptable salt thereof that is administered on each day of dosing is about 0.5 mg to about 100 mg. In some embodiments, the amount of Compound I-263a or a pharmaceutically acceptable salt thereof that is administered on each day of dosing is about 0.5 mg to about 50 mg. In some embodiments, the amount of Compound I-263a or a pharmaceutically acceptable salt thereof that is administered on each day of dosing is about 0.5 mg to about 10 mg. In some embodiments, the amount of Compound I-263a or a pharmaceutically acceptable salt thereof that is administered on each day of dosing is about 0.5 mg to about 5 mg. In some embodiments, the amount of Compound I-263a or a pharmaceutically acceptable salt thereof that is administered on each day of dosing is about 1 mg to about 3 mg. In some embodiments, the amount of Compound I-263a or a pharmaceutically acceptable salt thereof that is administered on each day of dosing is about 2 mg to about 5 mg. In some embodiments, the amount of Compound I-263a or a pharmaceutically acceptable salt thereof that is administered on each day of dosing is about 5 mg to about 10 mg. In some embodiments, the amount of Compound I-263a or a pharmaceutically acceptable salt thereof that is administered on each day of dosing is about 5 mg to about 15 mg. In some embodiments, the amount of Compound I-263a or a pharmaceutically acceptable salt thereof that is administered on each day of dosing is about 10 mg to about 20 mg. In some embodiments, the amount of Compound I-263a or a pharmaceutically acceptable salt thereof that is administered on each day of dosing is about 15 mg to about 25 mg. In some embodiments, the amount of Compound I-263a or a pharmaceutically acceptable salt thereof that is administered on each day of dosing is between about 20 mg to about 30 mg. In some embodiments, the amount of Compound I-263a or a pharmaceutically acceptable salt thereof that is administered on each day of dosing is between about 25 mg to about 35 mg. In some embodiments, the amount of Compound I-263a or a pharmaceutically acceptable salt thereof that is administered on each day of dosing is between about 30 mg to about 40 mg. In some embodiments, the amount of Compound I-263a or a pharmaceutically acceptable salt thereof that is administered on each day of dosing is between about 35 mg to about 45 mg. In some embodiments, the amount of Compound I-263 a or a pharmaceutically acceptable salt thereof that is administered on each day of dosing is between about 40 mg to about 50 mg. In some embodiments, the amount of Compound I-263a or a pharmaceutically acceptable salt thereof that is administered on each day of dosing is between about 55 mg to about 65 mg. In some embodiments, the amount of Compound I-263a or a pharmaceutically acceptable salt thereof that is administered on each day of dosing is between about 50 mg to about 100 mg. In some embodiments, the amount of Compound I-263a or a pharmaceutically acceptable salt thereof that is administered on each day of dosing is between about 90 mg to about 150 mg. In some embodiments, the amount of Compound I-263a or a pharmaceutically acceptable salt thereof that is administered on each day of dosing is between about 140 mg to about 200 mg. In some embodiments, the amount of Compound I-263a or a pharmaceutically acceptable salt thereof that is administered on each day of dosing is between about 3 mg to about 160 mg. In some embodiments, the amount of Compound I-263a or a pharmaceutically acceptable salt thereof that is administered on each day of dosing is about 0.5 mg. In some embodiments, the amount of Compound I-263a or a pharmaceutically acceptable salt thereof that is administered on each day of dosing is about 1 mg. In some embodiments, the amount of Compound I-263a or a pharmaceutically acceptable salt thereof that is administered on each day of dosing is about 2 mg. In some embodiments, the amount of Compound I-263a or a pharmaceutically acceptable salt thereof that is administered on each day of dosing is about 3 mg. In some embodiments, the amount of Compound I-263a or a pharmaceutically acceptable salt thereof that is administered on each day of dosing is about 4 mg. In some embodiments, the amount of Compound I-263a or a pharmaceutically acceptable salt thereof that is administered on each day of dosing is about 6 mg. In some embodiments, the amount of Compound I-263a or a pharmaceutically acceptable salt thereof that is administered on each day of dosing is about 8 mg. In some embodiments, the amount of Compound I-263a or a pharmaceutically acceptable salt thereof that is administered on each day of dosing is about 10 mg. In some embodiments, the amount of Compound I-263a or a pharmaceutically acceptable salt thereof that is administered on each day of dosing is about 12 mg. All dosing amounts refer to the amount of Compound I-263a administered, and do not include the weight amount of any pharmaceutically acceptable salt.

In some embodiments, the amount of Compound I-263a or a pharmaceutically acceptable salt thereof that is administered on each day of dosing is about 1 mg. In some embodiments, the amount of Compound I-263a or a pharmaceutically acceptable salt thereof that is administered on each day of dosing is about 3 mg. In some embodiments, the amount of Compound I-263a or a pharmaceutically acceptable salt thereof that is administered on each day of dosing is about 6 mg. In some embodiments, the amount of Compound I-263a or a pharmaceutically acceptable salt thereof that is administered on each day of dosing is about 10 mg. In some embodiments, the amount of Compound I-263a or a pharmaceutically acceptable salt thereof that is administered on each day of dosing is about 15 mg. In some embodiments, the amount of Compound I-263a or a pharmaceutically acceptable salt thereof that is administered on each day of dosing is about 25 mg. In some embodiments, the amount of Compound I-263a or a pharmaceutically acceptable salt thereof that is administered on each day of dosing is about 40 mg. In some embodiments, the amount of Compound I-263a or a pharmaceutically acceptable salt thereof that is administered on each day of dosing is about 50 mg. In some embodiments, the amount of Compound I-263a or a pharmaceutically acceptable salt thereof that is administered on each day of dosing is about 60 mg. In some embodiments, the amount of Compound I-263a or a pharmaceutically acceptable salt thereof that is administered on each day of dosing is about 70 mg. In some embodiments, the amount of Compound I-263a or a pharmaceutically acceptable salt thereof that is administered on each day of dosing is about 80 mg. In some embodiments, the amount of Compound I-263a or a pharmaceutically acceptable salt thereof that is administered on each day of dosing is about 90 mg. In some embodiments, the amount of Compound I-263a or a pharmaceutically acceptable salt thereof that is administered on each day of dosing is about 100 mg. In some embodiments, the amount of Compound I-263a or a pharmaceutically acceptable salt thereof that is administered on each day of dosing is about 110 mg. In some embodiments, the amount of Compound I-263a or a pharmaceutically acceptable salt thereof that is administered on each day of dosing is about 120 mg. In some embodiments, the amount of Compound I-263a or a pharmaceutically acceptable salt thereof that is administered on each day of dosing is about 130 mg. In some embodiments, the amount of Compound I-263a or a pharmaceutically acceptable salt thereof that is administered on each day of dosing is about 140 mg. In some embodiments, the amount of Compound I-263a or a pharmaceutically acceptable salt thereof that is administered on each day of dosing is about 150 mg. In some embodiments, the amount of Compound I-263a or a pharmaceutically acceptable salt thereof that is administered on each day of dosing is about 160 mg. In some embodiments, the amount of Compound I-263a or a pharmaceutically acceptable salt thereof that is administered on each day of dosing is about 170 mg. In some embodiments, the amount of Compound I-263a or a pharmaceutically acceptable salt thereof that is administered on each day of dosing is about 180 mg. In some embodiments, the amount of Compound I-263a or a pharmaceutically acceptable salt thereof that is administered on each day of dosing is about 190 mg. In some embodiments, the amount of Compound I-263a or a pharmaceutically acceptable salt thereof that is administered on each day of dosing is about 200 mg. In some embodiments, the amount of Compound I-263a or a pharmaceutically acceptable salt thereof that is administered on each day of dosing is about 210 mg. In some embodiments, the amount of Compound I-263a or a pharmaceutically acceptable salt thereof that is administered on each day of dosing is about 220 mg. In some embodiments, the amount of Compound I-263a or a pharmaceutically acceptable salt thereof that is administered on each day of dosing is about 230 mg. In some embodiments, the amount of Compound I-263a or a pharmaceutically acceptable salt thereof that is administered on each day of dosing is about 240 mg. In some embodiments, the amount of Compound I-263a or a pharmaceutically acceptable salt thereof that is administered on each day of dosing is about 250 mg.

In some embodiments, the amount of Compound I-263a or a pharmaceutically acceptable salt thereof that is administered on each day of dosing is about 3 mg, 6 mg, 10 , mg, 15 mg, 25 mg, 40 mg, 60 mg, 90 mg, or 120 mg.

In some embodiments, the amount of Compound I-263a or a pharmaceutically acceptable salt thereof that is administered on each day of dosing is about 0.5 mg/kg to about 200 mg/kg. In some embodiments, the amount of Compound I-263a or a pharmaceutically acceptable salt thereof that is administered on each day of dosing is about 0.5 mg/kg to about 100 mg/kg. In some embodiments, the amount of Compound I-263a or a pharmaceutically acceptable salt thereof that is administered on each day of dosing is about 0.5 mg/kg to about 50 mg/kg. In some embodiments, the amount of Compound I-263a or a pharmaceutically acceptable salt thereof that is administered on each day of dosing is about 0.5 mg/kg to about 10 mg/kg. In some embodiments, the amount of Compound I-263 a or a pharmaceutically acceptable salt thereof that is administered on each day of dosing is about 0.5 mg/kg to about 5 mg/kg. In some embodiments, the amount of Compound I-263a or a pharmaceutically acceptable salt thereof that is administered on each day of dosing is about 1 mg/kg to about 3 mg/kg. In some embodiments, the amount of Compound I-263a or a pharmaceutically acceptable salt thereof that is administered on each day of dosing is about 2 mg/kg to about 5 mg/kg. In some embodiments, the amount of Compound I-263a or a pharmaceutically acceptable salt thereof that is administered on each day of dosing is about 5 mg/kg to about 10 mg/kg. In some embodiments, the amount of Compound I-263a or a pharmaceutically acceptable salt thereof that is administered on each day of dosing is about 5 mg/kg to about 15 mg/kg. In some embodiments, the amount of Compound I-263a or a pharmaceutically acceptable salt thereof that is administered on each day of dosing is about 10 mg/kg to about 20 mg/kg. In some embodiments, the amount of Compound I-263a or a pharmaceutically acceptable salt thereof that is administered on each day of dosing is about 15 mg/kg to about 25 mg/kg. In some embodiments, the amount of Compound I-263a or a pharmaceutically acceptable salt thereof that is administered on each day of dosing is between about 20 mg/kg to about 30 mg/kg. In some embodiments, the amount of Compound I-263a or a pharmaceutically acceptable salt thereof that is administered on each day of dosing is between about 25 mg/kg to about 35 mg/kg. In some embodiments, the amount of Compound I-263a or a pharmaceutically acceptable salt thereof that is administered on each day of dosing is between about 30 mg/kg to about 40 mg/kg. In some embodiments, the amount of Compound I-263a or a pharmaceutically acceptable salt thereof that is administered on each day of dosing is between about 35 mg/kg to about 45 mg/kg. In some embodiments, the amount of Compound I-263a or a pharmaceutically acceptable salt thereof that is administered on each day of dosing is between about 40 mg/kg to about 50 mg/kg. In some embodiments, the amount of Compound I-263a or a pharmaceutically acceptable salt thereof that is administered on each day of dosing is between about 55 mg/kg to about 65 mg/kg. In some embodiments, the amount of Compound I-263a or a pharmaceutically acceptable salt thereof that is administered on each day of dosing is between about 50 mg/kg to about 100 mg/kg. In some embodiments, the amount of Compound I-263a or a pharmaceutically acceptable salt thereof that is administered on each day of dosing is between about 90 mg/kg to about 150 mg/kg. In some embodiments, the amount of Compound I-263a or a pharmaceutically acceptable salt thereof that is administered on each day of dosing is between about 140 mg/kg to about 200 mg/kg. In some embodiments, the amount of Compound I-263a or a pharmaceutically acceptable salt thereof that is administered on each day of dosing is about 0.5 mg/kg. In some embodiments, the amount of Compound I-263a or a pharmaceutically acceptable salt thereof that is administered on each day of dosing is about 1 mg/kg. In some embodiments, the amount of Compound I-263a or a pharmaceutically acceptable salt thereof that is administered on each day of dosing is about 2 mg/kg. In some embodiments, the amount of Compound I-263a or a pharmaceutically acceptable salt thereof that is administered on each day of dosing is about 3 mg/kg. In some embodiments, the amount of Compound I-263a or a pharmaceutically acceptable salt thereof that is administered on each day of dosing is about 2.5 mg/kg. In some embodiments, the amount of Compound I-263a or a pharmaceutically acceptable salt thereof that is administered on each day of dosing is about 4 mg/kg. In some embodiments, the amount of Compound I-263a or a pharmaceutically acceptable salt thereof that is administered on each day of dosing is about 5 mg/kg. In some embodiments, the amount of Compound I-263a or a pharmaceutically acceptable salt thereof that is administered on each day of dosing is about 6 mg/kg. In some embodiments, the amount of Compound I-263a or a pharmaceutically acceptable salt thereof that is administered on each day of dosing is about 7.5 mg/kg. In some embodiments, the amount of Compound I-263a or a pharmaceutically acceptable salt thereof that is administered on each day of dosing is about 8 mg/kg. In some embodiments, the amount of Compound I-263a or a pharmaceutically acceptable salt thereof that is administered on each day of dosing is about 10 mg/kg. In some embodiments, the amount of Compound I-263a or a pharmaceutically acceptable salt thereof that is administered on each day of dosing is about 12 mg/kg. In some embodiments, the amount of Compound I-263a or a pharmaceutically acceptable salt thereof that is administered on each day of dosing is about 12.5 mg/kg. In some embodiments, the amount of Compound I-263a or a pharmaceutically acceptable salt thereof that is administered on each day of dosing is about 15 mg/kg. In some embodiments, the amount of Compound I-263a or a pharmaceutically acceptable salt thereof that is administered on each day of dosing is about 17.5 mg/kg. In some embodiments, the amount of Compound I-263a or a pharmaceutically acceptable salt thereof that is administered on each day of dosing is about 20 mg/kg. In some embodiments, the amount of Compound I-263a or a pharmaceutically acceptable salt thereof that is administered on each day of dosing is about 25 mg/kg. In some embodiments, the amount of Compound I-263a or a pharmaceutically acceptable salt thereof that is administered on each day of dosing is about 30 mg/kg. All dosing amounts refer to the amount of Compound I-263a administered, and do not include the weight amount of any pharmaceutically acceptable salt.

In some embodiments, the amount of Compound I-263a or a pharmaceutically acceptable salt thereof that is administered on each day of dosing is about 1 mg/kg. In some embodiments, the amount of Compound I-263a or a pharmaceutically acceptable salt thereof that is administered on each day of dosing is about 3 mg/kg. In some embodiments, the amount of Compound I-263a or a pharmaceutically acceptable salt thereof that is administered on each day of dosing is about 6 mg/kg. In some embodiments, the amount of Compound I-263a or a pharmaceutically acceptable salt thereof that is administered on each day of dosing is about 7.5 mg/kg. In some embodiments, the amount of Compound I-263a or a pharmaceutically acceptable salt thereof that is administered on each day of dosing is about 10 mg/kg. In some embodiments, the amount of Compound I-263a or a pharmaceutically acceptable salt thereof that is administered on each day of dosing is about 15 mg/kg. In some embodiments, the amount of Compound I-263a or a pharmaceutically acceptable salt thereof that is administered on each day of dosing is about 25 mg/kg. In some embodiments, the amount of Compound I-263a or a pharmaceutically acceptable salt thereof that is administered on each day of dosing is about 40 mg/kg. In some embodiments, the amount of Compound I-263a or a pharmaceutically acceptable salt thereof that is administered on each day of dosing is about 60 mg/kg. In some embodiments, the amount of Compound I-263a or a pharmaceutically acceptable salt thereof that is administered on each day of dosing is about 90 mg/kg. In some embodiments, the amount of Compound I-263a or a pharmaceutically acceptable salt thereof that is administered on each day of dosing is about 120 mg/kg. In some embodiments, the amount of Compound I-263a or a pharmaceutically acceptable salt thereof that is administered on each day of dosing is about 160 mg/kg.

In some embodiments, the anti-CD38 antibody is administered on a daily schedule. In some embodiments, the anti-CD38 antibody is administered every other day. In some embodiments, the anti-CD38 antibody is administered once every three days. In some embodiments, the anti-CD38 antibody is administered on a twice-weekly schedule. In some embodiments, the anti-CD38 antibody is administered on a three times a week schedule. In some embodiments, the anti-CD38 antibody is administered on a weekly schedule. In some embodiments, the anti-CD38 antibody is administered on a once every two weeks schedule. In some embodiments, the anti-CD38 antibody is administered on a once every three weeks schedule. In some embodiments, the anti-CD38 antibody is administered on a once every four weeks schedule. In some embodiments, the anti-CD38 antibody is administered on a once every eight weeks schedule.

In some embodiments, the anti-CD38 antibody is administered at least 3 times on alternate days within a 7-day cycle. In some embodiments, the anti-CD38 antibody is administered on day 1 and day 4 of a 7-day cycle. In some embodiments, the anti-CD38 antibody is administered on consecutive days in a 7-day cycle followed by an intermission. In some embodiments, the anti-CD38 antibody is administered for 2 consecutive days followed by an intermission of 5 consecutive days for at least one 7-day cycle. In some embodiments, the anti-CD38 antibody is administered for 3 consecutive days followed by an intermission of 4 consecutive days for at least one 7-day cycle. In some embodiments, the anti-CD38 antibody is administered for 4 consecutive days followed by an intermission of 3 consecutive days for at least one 7-day cycle. In some embodiments, the anti-CD38 antibody is administered for 5 consecutive days followed by an intermission of 2 consecutive days for at least one 7-day cycle.

Dosing schedules include, for example, administering the anti-CD38 antibody once during a treatment schedule, e.g., on day 1 of a 21 day cycle or day 1 of a 28 day cycle, twice during a treatment cycle, e.g., on days 1 and 15 of a 21 day cycle or on days 1 and 15 of a 28 day cycle, three times during a treatment cycle, e.g., on days 1, 8 and 15 of a 21 day cycle or on days 1, 8 and 15 of a 28 day cycle, four times during a treatment cycle, e.g., on days 1, 4, 8, and 11 of a 14 days cycle, days 0, 3, 7, and 10 of a 14 day cycle, on days 1, 4, 8, and 11 of a 21 day cycle, on days 1, 8, 15, and 22 of a 28 day cycle, or on days 1, 4, 8, and 11 of a 28 day cycle, six times during a treatment cycle, e.g., on days 1, 4, 8, 11, 15, and 18 of a 21 day cycle or on days 1, 4, 8, 11, 15, and 18 of a 28 day cycle, eight times during a treatment cycle, e.g. days 1, 4, 8, 11, 15, 18, 22 and 25 of a 28 day cycle or days 1, 4, 8, 11, 15, 18, 22 and 25 of a 28 day, ten times during a 35 day cycle, e.g. days 1, 4, 8, 11, 15, 18, 22, 25, 29 and 33 of a 35 day cycle. Day 0 of a treating cycle is one day immediately before the day that the treatment cycle starts. Other dosage schedules are encompassed by the present invention.

In some embodiments, the anti-CD38 antibody is administered within a 14-day cycle. In some embodiments, the anti-CD38 antibody is administered twice weekly on days on 0, 3, 7, and 10 of the 14 day cycle. In some embodiments, the anti-CD38 antibody is administered twice weekly on days on 1, 4, 8, and 11 of the 14 day cycle.

In some embodiments, the anti-CD38 antibody is administered within a 21 day cycle. In some embodiments, the anti-CD38 antibody is administered twice weekly on days on 1, 4, 8, 11, 15, and 18 of the 21 day cycle.

In some embodiments, the anti-CD38 antibody is administered within a 28 day cycle. In some embodiments, the anti-CD38 antibody is administered twice weekly on days on 1, 4, 8, 11, 15, and 18 of the 28 day cycle. In some embodiments, the anti-CD38 antibody is administered twice weekly on days on 1, 8, 15, and 22 of the 28 day cycle. In some embodiments, the anti-CD38 antibody is administered once weekly on days on 1, 8, 15, and 22 of the 28 day cycle. In some embodiments, the anti-CD38 antibody is administered once weekly in cycles 1 and 2, followed by every 2 weeks in cycles 3 through 6, then monthly. In some embodiments, the anti-CD38 antibody is administered once weekly in cycles 1 and 2 of 28-day cycle, followed by every 2 weeks in cycles 3 through 6, then monthly. In some embodiments, the anti-CD38 antibody is administered once weekly on days on 1 and 15 of the 28 day cycle. In some embodiments, the anti-CD38 antibody is administered once on day 1 of the 28 day cycle.

In some embodiments, the anti-CD38 antibody is administered within a 35 day cycle. In some embodiments, the anti-CD38 antibody is administered twice weekly on days on 1, 4, 8, 11, 15, 18, 22, 25, 29 and 33 of the 35 day cycle.

In some embodiments, the anti-CD38 antibody is administered on days 1, 8, 15, and 22 of a 28 day cycle.

In some embodiments, the anti-CD38 antibody is administered on days 1 and 15 of a 28 day cycle.

In some embodiments, the anti-CD38 antibody is administered on day 1 of a 28 day cycle.

In some embodiments, the anti-CD38 antibody is administered in a loading dose or induction schedule for anywhere from 1 to 54 weeks followed by a maintenance dose or consolidation schedule thereafter.

In some embodiments, the anti-CD38 antibody is administered by subcutaneous injection. In some embodiments, the anti-CD38 antibody is administered by intravenous infusion followed by one or more subsequent subcutaneous injections. In some embodiments, the intravenous infusion and one or more subsequent subcutaneous injections are administered according to the dosing schedules and methods disclosed herein.

In some embodiments, the amount of the anti-CD38 antibody that is administered on each day of dosing is about 0.005 mg/kg to about 100 mg/kg. In some embodiments, the amount of the anti-CD38 antibody that is administered on each day of dosing is about 0.05 mg/kg to about 100 mg/kg. In some embodiments, the amount of the anti-CD38 antibody that is administered on each day of dosing is about 0.05 mg/kg to about 30 mg/kg. In some embodiments, the amount of the anti-CD38 antibody that is administered on each day of dosing is about 0.5 mg/kg to about 100 mg/kg. In some embodiments, the amount of the anti-CD38 antibody that is administered on each day of dosing is about 0.5 mg/kg to about 50 mg/kg. In some embodiments, the amount of the anti-CD38 antibody that is administered on each day of dosing is about 0.5 mg/kg to about 25 mg/kg. In some embodiments, the amount of the anti-CD38 antibody that is administered on each day of dosing is about 0.5 mg/kg to about 10 mg/kg. In some embodiments, the amount of the anti-CD38 antibody that is administered on each day of dosing is about 0.5 mg/kg to about 5 mg/kg. In some embodiments, the amount of the anti-CD38 antibody that is administered on each day of dosing is about 5 mg/kg to about 100 mg/kg. In some embodiments, the amount of the anti-CD38 antibody that is administered on each day of dosing is about 10 mg/kg to about 100 mg/kg. In some embodiments, the amount of the anti-CD38 antibody that is administered on each day of dosing is about 50 mg/kg to about 100 mg/kg.

In some embodiments, the amount of the anti-CD38 antibody that is administered on each day of dosing is about 1 mg/kg. In some embodiments, the amount of the anti-CD38 antibody that is administered on each day of dosing is about 2 mg/kg. In some embodiments, the amount of the anti-CD38 antibody that is administered on each day of dosing is about 2.5 mg/kg. In some embodiments, the amount of the anti-CD38 antibody that is administered on each day of dosing is about 3 mg/kg. In some embodiments, the amount of the anti-CD38 antibody that is administered on each day of dosing is about 4 mg/kg. In some embodiments, the amount of the anti-CD38 antibody that is administered on each day of dosing is about 5 mg/kg. In some embodiments, the amount of the anti-CD38 antibody that is administered on each day of dosing is about 6 mg/kg. In some embodiments, the amount of the anti-CD38 antibody that is administered on each day of dosing is about 7 mg/kg. In some embodiments, the amount of the anti-CD38 antibody that is administered on each day of dosing is about 7.5 mg/kg. In some embodiments, the amount of the anti-CD38 antibody that is administered on each day of dosing is about 8 mg/kg. In some embodiments, the amount of the anti-CD38 antibody that is administered on each day of dosing is about 9 mg/kg. In some embodiments, the amount of the anti-CD38 antibody that is administered on each day of dosing is about 10 mg/kg. In some embodiments, the amount of the anti-CD38 antibody that is administered on each day of dosing is about 15 mg/kg. In some embodiments, the amount of the anti-CD38 antibody that is administered on each day of dosing is about 16 mg/kg. In some embodiments, the amount of the anti-CD38 antibody that is administered on each day of dosing is about 17 mg/kg. In some embodiments, the amount of the anti-CD38 antibody that is administered on each day of dosing is about 18 mg/kg. In some embodiments, the amount of the anti-CD38 antibody that is administered on each day of dosing is about 19 mg/kg. In some embodiments, the amount of the anti-CD38 antibody that is administered on each day of dosing is about 20 mg/kg. In some embodiments, the amount of the anti-CD38 antibody that is administered on each day of dosing is about 21 mg/kg. In some embodiments, the amount of the anti-CD38 antibody that is administered on each day of dosing is about 22 mg/kg. In some embodiments, the amount of the anti-CD38 antibody that is administered on each day of dosing is about 23 mg/kg. In some embodiments, the amount of the anti-CD38 antibody that is administered on each day of dosing is about 24 mg/kg. In some embodiments, the amount of the anti-CD38 antibody that is administered on each day of dosing is about 25 mg/kg. In some embodiments, the amount of the anti-CD38 antibody that is administered on each day of dosing is about 30 mg/kg. In some embodiments, the amount of the anti-CD38 antibody that is administered on each day of dosing is about 40 mg/kg. In some embodiments, the amount of the anti-CD38 antibody that is administered on each day of dosing is about 50 mg/kg. In some embodiments, the amount of the anti-CD38 antibody that is administered on each day of dosing is about 60 mg/kg. In some embodiments, the amount of the anti-CD38 antibody that is administered on each day of dosing is about 70 mg/kg. In some embodiments, the amount of the anti-CD38 antibody that is administered on each day of dosing is about 80 mg/kg. In some embodiments, the amount of the anti-CD38 antibody that is administered on each day of dosing is about 90 mg/kg. In some embodiments, the amount of the anti-CD38 antibody that is administered on each day of dosing is about 100 mg/kg.

In some embodiments, the amount of the anti-CD38 antibody that is administered on each day of dosing is a fixed unit dose, for example, 50, 100, 200, 300, 400, 500, 600, 700, 800, 900, 1000, 1100, 1200, 1300, 1400, 1500, 1600, 1700, 1800, 1900, or 2000 mg. In some embodiments, the amount of the anti-CD38 antibody that is administered on each day of dosing is based on the patient’s body surface area (BSA), e.g., 500, 400, 300, 250, 200, or 100 mg/m². As used herein, body surface area is calculated using a standard nomogram, e.g.,

$$\text{BSA} \left({\text{m}}^{2}\right) = \sqrt{\frac{\text{Ht} \left(\text{cm}\right) \times \text{Wt} \left(\text{kg}\right)}{3600}} \text{or} \text{BSA} = \sqrt{\frac{\text{Ht} \left(\text{in}\right) \times \text{Wt} \left(\text{lb}\right)}{3131}}$$

In some embodiments, the anti-CD38 antibody is daratumumab. The daratumumab is administered in an injectable formulation that comprises daratumumab, or a pharmaceutically acceptable salt thereof, and optionally hyaluronidase-fihj. When hyaluronidase-fijh is administered with daratumumab, the hyaluronidase-fijh is administered in an amount of about 1,500 to about 2,500 units/mL for every 120 mg of daratumumab. In some embodiments, the hyaluronidase-fijh is administered in an amount of about 2,000 units/mL for every 120 mg of daratumumab. In some embodiments, the amount of daratumumab, or a pharmaceutically acceptable salt thereof that is administered on each day of dosing is about 0.5 mg/kg to about 1000 mg/kg. In some embodiments, the amount of daratumumab, or a pharmaceutically acceptable salt thereof that is administered on each day of dosing is about 0.5 mg/kg to about 200 mg/kg. In some embodiments, the amount of daratumumab, or a pharmaceutically acceptable salt thereof that is administered on each day of dosing is about 0.5 mg/kg to about 100 mg/kg. In some embodiments, the amount of daratumumab, or a pharmaceutically acceptable salt thereof that is administered on each day of dosing is about 0.5 mg/kg to about 50 mg/kg. In some embodiments, the amount of daratumumab, or a pharmaceutically acceptable salt thereof that is administered on each day of dosing is about 0.5 mg/kg to about 25 mg/kg. In some embodiments, the amount of daratumumab, or a pharmaceutically acceptable salt thereof that is administered on each day of dosing is about 0.5 mg/kg to about 20 mg/kg. In some embodiments, the amount of daratumumab, or a pharmaceutically acceptable salt thereof that is administered on each day of dosing is about 0.5 mg/kg to about 10 mg/kg. In some embodiments, the amount of daratumumab, or a pharmaceutically acceptable salt thereof that is administered on each day of dosing is about 0.5 mg/kg to about 5 mg/kg. In some embodiments, the amount of daratumumab, or a pharmaceutically acceptable salt thereof that is administered on each day of dosing is about 1 mg/kg. In some embodiments, the amount of daratumumab, or a pharmaceutically acceptable salt thereof that is administered on each day of dosing is about 2.5 mg/kg. In some embodiments, the amount of daratumumab, or a pharmaceutically acceptable salt thereof that is administered on each day of dosing is about 5 mg/kg. In some embodiments, the amount of daratumumab, or a pharmaceutically acceptable salt thereof that is administered on each day of dosing is about 7.5 mg/kg. In some embodiments, the amount of daratumumab, or a pharmaceutically acceptable salt thereof that is administered on each day of dosing is about 10 mg/kg. In some embodiments, the amount of daratumumab, or a pharmaceutically acceptable salt thereof that is administered on each day of dosing is about 12.5 mg/kg. In some embodiments, the amount of daratumumab, or a pharmaceutically acceptable salt thereof that is administered on each day of dosing is about 15 mg/kg. In some embodiments, the amount of daratumumab, or a pharmaceutically acceptable salt thereof that is administered on each day of dosing is about 16 mg/kg. In some embodiments, the amount of daratumumab, or a pharmaceutically acceptable salt thereof that is administered on each day of dosing is about 17.5 mg/kg. In some embodiments, the amount of daratumumab, or a pharmaceutically acceptable salt thereof that is administered on each day of dosing is about 20 mg/kg. In some embodiments, the amount of daratumumab, or a pharmaceutically acceptable salt thereof that is administered on each day of dosing is about 22.5 mg/kg. In some embodiments, the amount of daratumumab, or a pharmaceutically acceptable salt thereof that is administered on each day of dosing is about 25 mg/kg. In some embodiments, the amount of daratumumab, or a pharmaceutically acceptable salt thereof that is administered on each day of dosing is about 30 mg/kg. In some embodiments, the amount of daratumumab or a pharmaceutically acceptable salt thereof that is administered on each day of dosing is about 50 mg/kg. In some embodiments, the amount of daratumumab, or a pharmaceutically acceptable salt thereof that is administered on each day of dosing is about 100 mg/kg. In some embodiments, the amount of daratumumab, or a pharmaceutically acceptable salt thereof that is administered on each day of dosing is about 200 mg/kg. In some embodiments, the amount of daratumumab, or a pharmaceutically acceptable salt thereof that is administered on each day of dosing is about 500 mg/kg. In some embodiments, the amount of daratumumab, or a pharmaceutically acceptable salt thereof that is administered on each day of dosing is about 1000 mg/kg. All dosing amounts refer to the amount of daratumumab, or a pharmaceutically acceptable salt thereof administered, and do not include the weight amount of any pharmaceutically acceptable salt. Other embodiments include administration of amounts of daratumumab in conjunction with hyaluronidase-fijh, where the hyaluronidase-fijh is provided in an amount of about 2000 units/mL per 120 mg of daratumumab.

In some embodiments, the amount of daratumumab, or a pharmaceutically acceptable salt thereof that is administered on each day of dosing is about 2.5 mg/kg. In some embodiments, the amount of daratumumab, or a pharmaceutically acceptable salt thereof that is administered on each day of dosing is about 7.5 mg/kg. In some embodiments, the amount of daratumumab, or a pharmaceutically acceptable salt thereof that is administered on each day of dosing is about 16 mg/kg. In some embodiments, the amount of daratumumab, or a pharmaceutically acceptable salt thereof that is administered on each day of dosing is about 20 mg/kg. Other embodiments include administration of amounts of daratumumab in conjunction with hyaluronidase-fijh, where the hyaluronidase-fijh is provided in an amount of about 2000 units/mL per 120 mg of daratumumab.

In some embodiments, the amount of daratumumab, or a pharmaceutically acceptable salt thereof that is administered on each day of dosing is from about 100 mg to about 2000 mg. In some embodiments, the amount of daratumumab, or a pharmaceutically acceptable salt thereof that is administered on each day of dosing is from about 200 mg to about 2000 mg. In some embodiments, the amount of daratumumab, or a pharmaceutically acceptable salt thereof that is administered on each day of dosing is from about 400 mg to about 2000 mg. In some embodiments, the amount of daratumumab, or a pharmaceutically acceptable salt thereof that is administered on each day of dosing is from about 600 mg to about 2000 mg. In some embodiments, the amount of daratumumab, or a pharmaceutically acceptable salt thereof that is administered on each day of dosing is from about 800 mg to about 2000 mg. In some embodiments, the amount of daratumumab, or a pharmaceutically acceptable salt thereof that is administered on each day of dosing is from about 1000 mg to about 2000 mg. In some embodiments, the amount of daratumumab, or a pharmaceutically acceptable salt thereof that is administered on each day of dosing is from about 1200 mg to about 2000 mg. In some embodiments, the amount of daratumumab, or a pharmaceutically acceptable salt thereof that is administered on each day of dosing is from about 1400 mg to about 2000 mg. In some embodiments, the amount of daratumumab, or a pharmaceutically acceptable salt thereof that is administered on each day of dosing is from about 1600 mg to about 2000 mg. In some embodiments, the amount of daratumumab, or a pharmaceutically acceptable salt thereof that is administered on each day of dosing is from about 1800 mg to about 2000 mg. In some embodiments, the amount of daratumumab, or a pharmaceutically acceptable salt thereof that is administered on each day of dosing is about 200 mg. In some embodiments, the amount of daratumumab, or a pharmaceutically acceptable salt thereof that is administered on each day of dosing is about 400 mg. In some embodiments, the amount of daratumumab, or a pharmaceutically acceptable salt thereof that is administered on each day of dosing is about 600 mg. In some embodiments, the amount of daratumumab, or a pharmaceutically acceptable salt thereof that is administered on each day of dosing is about 800 mg. In some embodiments, the amount of daratumumab, or a pharmaceutically acceptable salt thereof that is administered on each day of dosing is about 1000 mg. In some embodiments, the amount of daratumumab, or a pharmaceutically acceptable salt thereof that is administered on each day of dosing is about 1200 mg. In some embodiments, the amount of daratumumab, or a pharmaceutically acceptable salt thereof that is administered on each day of dosing is about 1400 mg. In some embodiments, the amount of daratumumab, or a pharmaceutically acceptable salt thereof that is administered on each day of dosing is about 1600 mg. In some embodiments, the amount of daratumumab, or a pharmaceutically acceptable salt thereof that is administered on each day of dosing is about 1800 mg. In some embodiments, the amount of daratumumab, or a pharmaceutically acceptable salt thereof that is administered on each day of dosing is about 2000 mg. Other embodiments include administration of amounts of daratumumab in conjunction with hyaluronidase-fijh, where the hyaluronidase-fijh is provided in an amount of about 2000 units/mL per 120 mg of daratumumab.

In some embodiments, the administration of daratumumab, or daratumumab and hyaluronidase-fihj, is in accordance with its prescribing information as approved by the health authorities, such as those issued by the FDA, or the EMA, which are incorporated here by their entirety.

In some embodiments, the anti-CD38 antibody is mezagitamab, or a pharmaceutically acceptable salt thereof. In some embodiments, the amount of mezagitamab, or a pharmaceutically acceptable salt thereof that is administered on each day of dosing is from about 10 mg to about 2000 mg. In some embodiments, the amount of mezagitamab, or a pharmaceutically acceptable salt thereof that is administered on each day of dosing is from about 10 mg to about 1500 mg. In some embodiments, the amount of mezagitamab, or a pharmaceutically acceptable salt thereof that is administered on each day of dosing is from about 10 mg to about 1000 mg. In some embodiments, the amount of mezagitamab, or a pharmaceutically acceptable salt thereof that is administered on each day of dosing is from about 100 mg to about 900 mg. In some embodiments, the amount of mezagitamab, or a pharmaceutically acceptable salt thereof that is administered on each day of dosing is from about 200 mg to about 800 mg. In some embodiments, the amount of mezagitamab, or a pharmaceutically acceptable salt thereof that is administered on each day of dosing is from about 300 mg to about 700 mg. In some embodiments, the amount of mezagitamab, or a pharmaceutically acceptable salt thereof that is administered on each day of dosing is from about 400 mg to about 700 mg. In some embodiments, the amount of mezagitamab, or a pharmaceutically acceptable salt thereof that is administered on each day of dosing is from about 500 mg to about 700 mg. In some embodiments, the amount of mezagitamab, or a pharmaceutically acceptable salt thereof that is administered on each day of dosing is about 10 mg. In some embodiments, the amount of mezagitamab, or a pharmaceutically acceptable salt thereof that is administered on each day of dosing is about 100 mg. In some embodiments, the amount of mezagitamab, or a pharmaceutically acceptable salt thereof that is administered on each day of dosing is about 200 mg. In some embodiments, the amount of mezagitamab, or a pharmaceutically acceptable salt thereof that is administered on each day of dosing is about 300 mg. In some embodiments, the amount of mezagitamab, or a pharmaceutically acceptable salt thereof that is administered on each day of dosing is about 400 mg. In some embodiments, the amount of mezagitamab, or a pharmaceutically acceptable salt thereof that is administered on each day of dosing is about 500 mg. In some embodiments, the amount of mezagitamab, or a pharmaceutically acceptable salt thereof that is administered on each day of dosing is about 600 mg. In some embodiments, the amount of mezagitamab, or a pharmaceutically acceptable salt thereof that is administered on each day of dosing is about 700 mg. In some embodiments, the amount of mezagitamab, or a pharmaceutically acceptable salt thereof that is administered on each day of dosing is about 800 mg. In some embodiments, the amount of mezagitamab, or a pharmaceutically acceptable salt thereof that is administered on each day of dosing is about 900 mg. In some embodiments, the amount of mezagitamab, or a pharmaceutically acceptable salt thereof that is administered on each day of dosing is about 1000 mg. In some embodiments, the amount of mezagitamab, or a pharmaceutically acceptable salt thereof that is administered on each day of dosing is about 1500 mg. In some embodiments, the amount of mezagitamab, or a pharmaceutically acceptable salt thereof that is administered on each day of dosing is about 2000 mg.

### Pharmaceutical Compositions

The SAE inhibitors and the anti-CD38 antibodies used in the methods and kits described herein can be formulated into pharmaceutical compositions suitable for administration. The pharmaceutical compositions may comprise pharmaceutically acceptable excipients. A pharmaceutically acceptable excipient, as used herein, includes, but are not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cyoprotectants, and/or bulking agents, as suited to the particular dosage form desired. Various excipients for formulating pharmaceutical compositions and techniques for preparing the composition are known in the art (see Remington: The Science and Practice of Pharmacy, 21^{st} Ed., A. R. Gennaro (Lippincott, Williams & Wilkins, Baltimore, MD), 2006; incorporated by reference in its entirety)

Any of the therapeutic agents described herein may be in the form of a pharmaceutically acceptable salt. In some embodiments, such salts are derived from inorganic or organic acids or bases. For reviews of suitable salts, see, e.g., Berge et al., J. Pharm. Sci., 1977, 66, 1-19 and Remington: The Science and Practice of Pharmacy, 20th Ed., A. Gennaro (ed.), Lippincott Williams & Wilkins (2000).

Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts, N-methyl-D-glucamine; and salts with amino acids such as arginine, lysine, and the like.

For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2-ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′-di(dehydroabietyl)-ethylenediamine), hydrobromide, hydrochloride, hydroxynaphthoate, iodide, isethionate (2-hydroxyethanesulfonate), lactate, lactobionate, malate, maleate, mandelate, mesylate (methanesulfonate), methylbromide, methylnitrate, methylsulfate, mucate, napsylate (2-naphthalenesulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′-dibenzylethylenediamine), chloroprocaine, choline, diethanolamine, ethylenediamine, meglumine (N-methylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis(salicylate), napadisylate (1,5-naphthalenedisulfonate), oxalate, pectinate, persulfate, phenylethylbarbiturate, picrate, propionate, thiocyanate, tosylate and undecanoate; organic cations benethamine (N-benzylphenethylamine), clemizole (1-p-chlorobenzyl-2-pyrrolildine-1′-ylmethylbenzimidazole), diethylamine, piperazine and tromethamine (tris(hydroxymethyl)aminomethane); and metallic cations barium and bismuth.

The pharmaceutical compositions may comprise pharmaceutically acceptable carriers. As used herein, “pharmaceutically acceptable carrier” refers to a material that is compatible with a recipient subject (a human) and is suitable for delivering an active agent to the target site without terminating the activity of the agent. The toxicity or adverse effects, if any, associated with the carrier preferably are commensurate with a reasonable risk/benefit ratio for the intended use of the active agent.

Pharmaceutically acceptable carriers that may be used in these compositions include ion exchangers, alumina, aluminum stearate, lecithin, serum proteins, such as human serum albumin, buffer substances such as phosphates or carbonates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions for use in the methods of the present disclosure may be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may contain stabilizers, pH modifiers, surfactants, solubilizing agents, bioavailability modifiers and combinations of these. These pharmaceutical compositions are formulated for pharmaceutical administration to a human being. Such compositions may be administered orally, parenterally, by inhalation spray, topically, rectally, nasally, buccally, vaginally or via an implanted reservoir. The term “parenteral” as used herein includes subcutaneous, intravenous, intraperitoneal, intramuscular, intra-articular, intra-synovial, intrasternal, intrathecal, intrahepatic, intralesional and intracranial injection or infusion techniques. In some embodiments, the compositions are administered orally, intravenously or subcutaneously. In some embodiments, the compositions are administered orally. In some embodiments, the compositions are administered intravenously. In some embodiments, the intravenous administration can be intravenous infusion or intravenous injection. In some embodiments, the compositions are administered by an intravenous infusion. In some embodiments, the compositions are administered by an intravenous injection. In some embodiments, the compositions are administered by subcutaneous injection. In some embodiments, the compositions are administered by intravenous infusion and then subsequently administered by subcutaneous injection. In another embodiment, the anti-CD38 antibody is coadministered with human hyaluronidase subcutaneously. These formulations may be designed to be short-acting, fast-releasing, or long-acting. Furthermore, the compositions may be administered in a local rather than systemic means, such as administration (e.g., by injection) at a tumor site.

Pharmaceutical formulations may be prepared as liquid suspensions or solutions using a liquid, such as an oil, water, an alcohol, and combinations of these. Solubilizing agents such as cyclodextrins may be included. Pharmaceutically suitable surfactants, suspending agents, or emulsifying agents, may be added for oral or parenteral administration. Suspensions may include oils, such as peanut oil, sesame oil, cottonseed oil, corn oil and olive oil. Suspension preparations may also contain esters of fatty acids such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol; ethers, such as poly(ethyleneglycol); petroleum hydrocarbons such as mineral oil and petrolatum; and water.

Sterile injectable forms of these pharmaceutical compositions may be aqueous or oleaginous suspensions. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer’s solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as sorbitan alkyl esters, such as Tweens or Spans, and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

These pharmaceutical compositions may be orally administered in any orally acceptable dosage form including capsules, tablets, aqueous suspensions or solutions. When aqueous suspensions are required for oral use, the active ingredient is combined with emulsifying and suspending agents. If desired, certain sweetening, flavoring or coloring agents may also be added. For oral administration in a capsule form, useful diluents include lactose and dried cornstarch. In the case of tablets for oral use, carriers that are commonly used include lactose and corn starch. Lubricating agents, such as magnesium stearate, are also typically added. Coatings may be used for a variety of purposes, e.g., to mask taste, to affect the site of dissolution or absorption, or to prolong drug action. Coatings may be applied to a tablet or to granulated particles for use in a capsule.

Alternatively, these pharmaceutical compositions may be administered in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

These pharmaceutical compositions may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract may be affected in a rectal suppository formulation (see above) or in a suitable enema formulation. Topically-transdermal patches may also be used. For topical applications, the pharmaceutical compositions may be formulated in a suitable ointment containing the active component suspended or dissolved in one or more carriers. Carriers for topical administration of the compounds of the present disclosure include mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active component(s) suspended or dissolved in one or more pharmaceutically acceptable carriers. Suitable carriers include mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions may also be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In one embodiment, a compound of formula I-263a is formulated as a solution for intravenous infusion. In some embodiments, a compound of formula I-263a is formulated in a solution with a buffering agent or a pH modifying agent, and a cyclodextrin, such as a beta-cyclodextrin. In one embodiment, the solution includes phosphoric acid and Captisol (betadex sulfobutyl ether sodium) in water. In some embodiments, a compound of formula I-263a is formulated in a solution containing 10 mg/mL of compound I-263a.

In some embodiments, a compound of formula I-263a is formulated as a drug product, wherein the drug product contains compound I-263a in a solution of phosphoric acid and Captisol (betadex sulfobutyl ether sodium) in water. In some embodiments, the drug product is packaged with a volume of 10 mL of compound I-263a sterile solution. In some embodiments, the drug product is packaged with a volume of 10.5 mL of compound I-263a sterile solution.

In some embodiments, mezagitamab is formulated as a drug product for subcutaneous injection. In some embodiments, mezagitamab is formulated as a drug product, wherein the drug product contains 100 mg mezagitamab in 1 mL of liquid solvent. In some embodiments, the liquid solvent is water.

In some embodiments, daratumumab and hyaluronidase-fihj is formulated as a drug product for subcutaneous injection. In some embodiments, daratumumab and hyaluronidase-fihj is formulated as a drug product, wherein the drug product contains L-histidine, L-histidine hydrochloride monohydrate, L-methionine, polysorbate, sorbitol, and water.

### Kits

In some embodiments, the SAE inhibitor or the anti-CD38 antibody described herein may be manufactured for inclusion in a kit. A “kit” is any article of manufacture (e.g., a package or container) comprising at least one reagent or chemotherapeutic agent. A kit for use in the methods herein may comprise an SAE inhibitor, such as a compound of formula I-263a or a pharmaceutically acceptable salt thereof. In some embodiments, the kit may further include an anti-CD38 antibody, and optionally one or more additional therapeutic agents. In some embodiments, the kit may include a compound of formula I-263a or a pharmaceutically acceptable salt thereof, an anti-CD38 antibody, and optionally one or more additional therapeutic agents. In some embodiments, the kit may include one or more SAE inhibitors or pharmaceutically acceptable salts thereof. In some embodiments, the kit may include one or more anti-CD38 antibodies.

In some embodiments, the present disclosure relates to a kit comprising a medicament for use in treating cancer or autoimmune disease in a patient in need of such treatment. The kit comprises a medicament comprising an SAE inhibitor, and instructions for administering the SAE inhibitor and an anti-CD38 antibody; or the kit comprises a medicament comprising an anti-CD38 antibody, and instructions for administering the anti-CD38 antibody and an SAE inhibitor. The kit may contain a medicament comprising an SAE inhibitor and an anti-CD38 antibody, and instructions for administering the SAE inhibitor and the anti-CD38 antibody, wherein the medicament is in single dosage form or in separate dosage forms. In some embodiments, the kit optionally comprises one or more additional therapeutic agents.

In some embodiments, a kit comprising an SAE inhibitor and an anti-CD38 antibody may further include another component or reagent. In some embodiments, a reagent in the kit may be a diluent for preparing the SAE inhibitor for administration. In some embodiments, a reagent in the kit may be a diluent for preparing the anti-CD38 antibody for administration. In some embodiments, a component in the kit may be a vessel for mixing the combination of the SAE inhibitor and the anti-CD38 antibody.

In another aspect, the present disclosure relates to a kit for treating cancer or autoimmune disease comprising at least one medicament comprising at least one dose of Compound I-263a or a pharmaceutically acceptable salt thereof, and at least one medicament comprising at least one dose of an anti-CD38 antibody, said kit for treating cancer further comprising dosing instructions for administering the medicaments for treatment of the patient in recognized need thereof.

In order that this present disclosure be more fully understood, the following examples are set forth. These examples are illustrative only and are not intended to limit the scope of the present disclosure in any way.

### EXAMPLES

### Example 1: Statistical Analysis of Treatment and Combination Effect for Tumor Growth in Subcutaneous Xenograft Models

The following statistical analysis methods were utilized in each of Studies 1-6.

### 1.1 Computing Growth Rates

Low tumor volumes can cause problems with the data analysis, so all measurements below 25 cubic mm (including 0 values) were excluded from the analysis. After this exclusion step, the tumor volume was assumed to follow an exponential growth model. More specifically, for a given animal and treatment group,

$${log}_{10} \left({V}_{i}\right) = a + b \times {t}_{i} + {ε}_{i}$$

where Vᵢ is the tumor volume at the i^{th} time point. Here, a is the initial log volume, b is the tumor growth rate, and tᵢ is the measurement time in days. εᵢ is the residual error term, which was assumed to be uncorrelated and drawn from a normal distribution.

This model was fit separately for each animal within each treatment group. If an animal was sacrificed or died early, but at least two unique time points were measured (including baseline), then the data up to that point was used to estimate the growth rate. If the animal had measurements at less than two time points, then the animal was automatically excluded from the analysis.

In rare cases, the estimated growth rate for one or more animals might be very different from the other animals within the same group. To make the analysis robust, an interval was defined with a width of 30 times the median absolute deviation of the estimated growth rates for a given group. The interval was centered at the median of the growth rates for the group. If the growth rate for any animal fell outside this interval, the growth rate was replaced with the value at the boundary of the interval.

### 1.2 Pairwise Comparisons and Interval Analysis

Pairwise comparisons were performed, where the mean growth rates were compared for various pairs of treatment conditions. The results were summarized using the growth rate inhibition (GRI). Suppose µ_{T} and µ_{C} are the mean tumor growth rates for the treatment and reference groups, respectively. Define

$$\text{Growth rate inhibition} = \frac{{μ}_{C} - {μ}_{T}}{{μ}_{V}} \times 100 \%$$

Here, µᵥ is the mean tumor growth rate for the vehicle group, which in most cases is the same as the reference group. However, when comparing two different active treatments, the vehicle group would be different from the reference group.

Interval analysis, if requested, involved a specified treatment group and time interval compared with another treatment group and time interval. For the interval analysis (also known as regrowth analysis), the calculations were similar to the pairwise comparisons, except that the growth rates were computed using only the data from the specified time periods. Statistical significance was also determined using an unpaired t-test.

### 1.3 Combination Analysis

Combination analysis, if requested, was performed to determine if there was a benefit from combining drug treatments. This analysis was also based on the estimated tumor growth rates. The measure of synergy was defined as

$$\text{Synergy score} = \frac{{μ}_{A B} - {μ}_{A} - {μ}_{B} + {μ}_{C o n t r o l}}{{μ}_{V}} \times 100 \%$$

Here, µ_{AB}, µ_{A}, µ_{B}, and µ_{Control} are the mean growth rates for the combination, drug A, drug B, and control groups, respectively. As before, µᵥ is the mean tumor growth rate for the vehicle group, which in most cases is the same as the control group. The standard error of the synergy score was calculated as the square root of the sum of squared standard errors across the four groups. The degrees of freedom were estimated using the Welch-Satterthwaite equation. A hypothesis test was performed to determine if the synergy score differed from 0. P-values were calculated by dividing the synergy score by its standard error and tested against a t-distribution (two-tailed) with the above-calculated degrees of freedom. A P value of less than 0.05 is statistically significant.

The combination results can be interpreted as follows. Statistically significant negative synergy scores indicate a synergistic combination (“Syn.”). Statistically significant positive synergy scores indicate a sub-additive combination (“Sub-add.”) when the combination performs better (i.e. has a lower growth rate) than the best performing single agent. Statistically significant positive synergy scores indicate an antagonistic combination (“Antag.”) when the combination performs worse than the best performing single agent. Scores that are not statistically significant should be considered additive (“Add.”).

### Study 1

Daudi is a human Burkitt’s Lymphoma cell line. A Daudi human xenograft tumor model was generated by subcutaneous inoculation with 2.0 ×x 10⁶ Daudi cells (cell suspension) in 6 weeks old female CB17 SCID mice (Charles River Laboratories, 251 Ballardvale St., Wilmington, MA) in the flank. When the mean tumor volume reached approximately 120 mm³, the animals were randomized into one vehicle control and three treatment groups (n=8/group). Mice were then dosed with 20% HPβCD or Compound I-263a and/or daratumumab over a 21-day period. Tumor growth and body weight were measured twice per week during the treatment and post-treatment periods, and mice were humanely euthanized once they had reached their humane endpoint.

A 1.725 mg/mL and a 0.862 mg/mL stock solution of I-263a was formulated weekly in 20% HPβCD and administered intravenously (IV) based on an average body weight using a 0.2 mL dosing volume. Final doses I-263a received were 7.5 mg/kg. I-263a was administered on a BIW schedule for 3 weeks (Days 1, 4, 8, 11, 15, and 18). Daratumumab (anti-CD38 antibody) (Myoderm Medical Supplies, 48 E Main St., Norristown, PA 19401) was purchased at the beginning of the study and stored at 4° C. according to manufacturer’s instructions. Final dose daratumumab received was 7.5 mg/kg based on average body weight. Daratumumab was administered intraperitoneally (IP) using a 0.1 mL dosing volume, on a BIW schedule for 3 weeks (Days, 1, 4, 8, 11, 15, and 18).

Tumors were measured twice weekly using Vernier calipers. Tumor volumes were calculated using standard equation: V = W² x L/2, where V = volume, W = width, and L = length for the tumor. When mean tumor volumes reached approximately 120 mm³, mice were randomized into control and treatment groups. Mice were randomized into 4 groups (n=8/group) and dosed with vehicle (20% HPβCD), I-263a, daratumumab, or the combination of I-263a plus daratumumab at doses and schedules described above. Tumor size and body weight were measured twice a week for the duration of the study. Mice were euthanized when their tumor volumes exceeded approximately 2000 mm³ or when an individual tumor exceeded the humane end-point for size (the length of the tumor exceeded 2 cm).

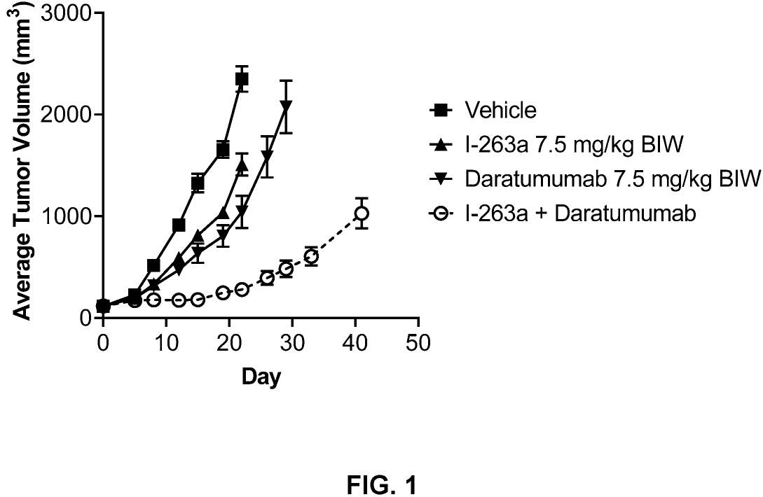

In the Daudi xenograft human Burkitt’s lymphoma subcutaneous tumor model, mice were inoculated, randomized on Day 0 (17 days post inoculation), and treatments began on Day 1 for all groups. I-263a was tested at 7.5 mg/kg administered IV on a BIW (twice per week) schedule for three weeks (Days 1, 4, 8, 11, 15, and 18). Daratumumab was tested at 7.5 mg/kg administered IP on a BIW schedule for three weeks (Days 1, 4, 8, 11, 15 and 18). In the combination treatment group, I-263a was administered first, followed immediately by the administration of daratumumab. One group served as the vehicle-treated group (Group 1) which received IV treatment with the vehicle for I-263a (20% HPβCD) on a BIW schedule for three weeks (Days 1, 4, 8, 11, 15, and 18). Statistical analyses were performed on data up to and including Day 22. Average tumor growth curves are shown in FIG. 1.

In a pairwise comparison with vehicle, single agent treatment with I-263a at 7.5 mg/kg BIW or daratumumab at 7.5 mg/kg BIW or the combination of both agents resulted in tumor growth inhibition relative to vehicle-treated tumors (p < 0.001). In a combination synergy analysis, I-263a at 7.5 mg/kg BIW plus daratumumab at 7.5 mg/kg BIW resulted in synergistic combination activity when compared to single-agent treatments (p < 0.01). These results demonstrate I-263a inhibits tumor growth in a Daudi human Burkitt’s Lymphoma mouse model and offers synergistic combination benefit when combined with daratumumab.

The treatment groups from Study 1 are shown in Table 1a. The combination effect for the treatment period is shown in Table 1b.

TABLE 1a

Combination of Compound I-263a and daratumumab in the Daudi xenograft model

| Study Group | Treatment | Dosing Regimen | Route | Tumor volume on Day 22 (± SEM) | GRI% | P-value |
|---|---|---|---|---|---|---|
| A | 20% HPβCD | BIW | IV | 2349.4 ± 124.9 | NA | NA |
| B | I-263a 7.5 mg/kg | BIW | IV | 1509.4 ± 109.1 | 15 | < 0.05 (0.01) |
| C | Daratumumab 7.5 mg/kg | BIW | IP | 1043.9 ± 159.1 | 29 | < 0.01 (0.001) |
| D | I-263a 7.5 mg/kg, Daratumumab 7.5 mg/kg | BIW, BIW | IV, IP | 280.9 ± 44.7 | 79 | < 0.001 |

TABLE 1b

Classification for in vivo combination of Compound I-263a and daratumumab in the Daudi xenograft model

| Treatment | Synergy score | SEM | P-value | Classification |
|---|---|---|---|---|
| I-263a 7.5 mg/kg, Daratumumab 7.5 mg/kg | -35 | 9 | <0.01 (0.001) | Synergistic |

### Study 2

A20-hCD38 is a mouse B-cell lymphoma cell line engineered to express the human CD38 cell surface protein. An A20-hCD38 syngeneic mouse tumor model was generated by subcutaneous inoculation with 5.0 × 10⁶ cells (cell suspension) in 6 weeks old female Balb/c mice (Jackson Laboratory, 10 Discovery Dr., Farmington, CT) in the flank. When the mean tumor volume reached approximately 70 mm³, the animals were randomized into one vehicle control and five treatment groups (n=8/group). Mice were dosed with 20% HPβCD or Compound I-263a or daratumumab over a 28-day period. Tumor growth and body weight were measured twice per week during the treatment and post-treatment periods, and mice were humanely euthanized once they had reached their humane endpoint.

A 1.575 mg/mL and a 0.788 mg/mL stock solution of I-263a was formulated weekly in 20% HPβCD and administered IV based on an average body weight using a 0.2 mL dosing volume. Final doses I-263a received were 15 mg/kg or 7.5 mg/kg, respectively. I-263a was administered on a QW or Q2W schedule for 4 weeks (Days 1, 8, 15, and 22). Daratumumab (anti-CD38 antibody) (Myoderm Medical Supplies, 48 E Main St., Norristown, PA 19401) was purchased at the beginning of the study and stored at 4° C. according to manufacturer’s instructions. Final dose daratumumab received was 20 mg/kg based on average body weight. Daratumumab was administered intraperitoneally (IP) using a 0.1 mL dosing volume, on a BIW schedule for 4 weeks (Days 1, 4, 8, 11, 15, 18, 22 and 25).

Tumors were measured twice weekly using Vernier calipers. Tumor volumes were calculated using standard equation: V = W² × L/2, where V = volume, W = width, and L = length for the tumor. When mean tumor volumes reached approximately 70 mm³, mice were randomized into control and treatment groups. Mice were randomized into 6 groups (n=8/group) and dosed with vehicle (20% HPβCD), I-263a, daratumumab, or the combination of I-263a plus daratumumab at doses and schedules as described above. Tumor size and body weight were measured twice a week for the duration of the study. Mice were euthanized when their tumor volumes exceeded approximately 2000 mm³ or when an individual tumor exceeded the humane end-point for size (the length of the tumor exceeded 2 cm).

In the A20-hCD38, mouse B-cell lymphoma line expressing human CD38 tumor model, mice were inoculated, randomized on Day 0 (5 days post inoculation), and treatments began on Day 1 for all groups.

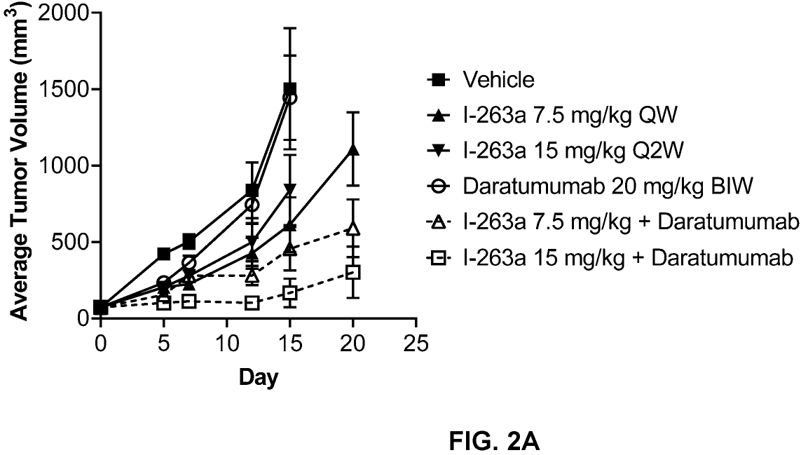

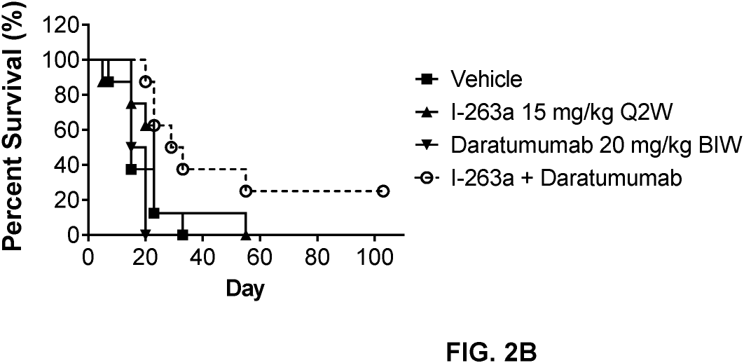

I-263a was tested at 7.5 mg/kg QW (once weekly; Days 1, 8, 15, and 22) or 15 mg/kg Q2W (every other week; Days 1 and 15) administered IV for four weeks. Daratumumab was tested at 20 mg/kg administered IP on a BIW (twice per week) schedule for four weeks (Days 1, 4, 8, 11, 15, 18, 22 and 25). In the combination treatment group, I-263a was administered first, followed immediately by the administration of daratumumab. One group served as the vehicle-treated group (Group 1) which received IV treatments with the vehicle for I-263a (20% HPβCD) on a QW schedule for four weeks (Days 1, 8, 15, and 22), though the group was removed on Day 15 due to excessive tumor volumes requiring euthanasia. Statistical analyses were performed on data up to and including Day 15. Average tumor growth curves are shown in FIG. 2A. Kaplan-Meier survival plots are shown in FIG. 2B.

In a pairwise comparison with vehicle, single agent treatment with I-263a at 7.5 mg/kg QW or 15 mg/kg Q2W or single agent daratumumab at 20 mg/kg BIW resulted in significant tumor growth inhibition relative to vehicle-treated tumors (p < 0.001). I-263a at 7.5 mg/kg QW or 15 mg/kg Q2W plus daratumumab at 20 mg/kg BIW resulted in significantly reduced tumor growth curves compared to vehicle treated mice (p < 0.001). In a combination synergy analysis I-263a at 7.5 mg/kg QW plus daratumumab at 20 mg/kg BIW resulted in additive combination benefit (p > 0.05) whereas I-263a at 15 mg/kg Q2W plus daratumumab at 20 mg/kg BIW resulted in synergistic combination activity (p < 0.05), when compared to single agent treatments. The combination of I-263a with daratumumab at 20 mg/kg resulted in significantly improved survival relative to I-263a along (p < 0.01) or daratumumab at 20 mg/kg alone (p < 0.001). P values for survival analysis were calculated by Weibull regression analysis.

The treatment groups from Study 2 are shown in Table 2a. The combination effect for the treatment period is shown in Table 2b.

TABLE 2a

Combination of Compound I-263a and daratumumab in an A20-hCD38 syngeneic model

| Study Group | Treatment | Dosing Regimen | Route | Tumor volume on Day 15 (± SEM) | GRI % | P-value |
|---|---|---|---|---|---|---|
| A | 20% HPβCD | BIW | IV | 1502.7 ± 397.2 | NA | NA |
| B | I-263a 7.5 mg/kg | QW | IV | 613.1 ± 179.3 | 37 | 0.095 |
| C | I-263a 15 mg/kg | Q2W | IV | 839.7 ± 229.7 | 24 | 0.239 |
| D | Daratumumab 20 mg/kg | BIW | IP | 1443.7 ± 276.4 | 3 | 0.885 |
| E | I-263a 7.5 mg/kg, Daratumumab 20 mg/kg | QW, BIW | IV, IP | 455.2 ± 141.4 | 54 | < 0.05 (0.032) |
| F | I-263a 15 mg/kg, Daratumumab 20 mg/kg | Q2W, BIW | IV, IP | 167.1 ± 93.3 | 78 | <0.01 (0.002) |

TABLE 2b

Classification for in vivo combination of Compound I-263a and daratumumab in the A20-hCD38 syngeneic model

| Treatment | Synergy score | SEM | P-value | Classification |
|---|---|---|---|---|
| I-263a 7.5 mg/kg, Daratumumab 20 mg/kg | -14 | 26 | 0.594 | Additive |
| I-263a 15 mg/kg, Daratumumab 20 mg/kg | -51 | 23 | < 0.05 (0.042) | Synergistic |

### Study 3

LP-1 is a human multiple myeloma cell line. A LP-1 human xenograft tumor model was generated by subcutaneous inoculation with 5.0 × 10³ LP-1 cells (cell suspension) in 7 weeks old female CB17 SCID mice (Charles River Laboratories, 251 Ballardvale St., Wilmington, MA) in the flank. When the mean tumor volume reached approximately 100 mm³, the animals were randomized into one vehicle control and seven treatment groups (n=8/group). Mice were then dosed with 20% HPβCD or Compound I-263a or daratumumab over a 35-day period. Tumor growth and body weight were measured twice per week during the treatment and post-treatment periods, and mice were humanely euthanized once they had reached their humane endpoint.

A 0.75 mg/mL stock solution of I-263a was formulated weekly in 20% HPβCD and administered IV based on an average body weight using a 0.2 mL dosing volume. Final dose I-263a received was 7.5 mg/kg. I-263a was administered on a BIW schedule for 5 weeks (Days 1, 4, 8, 11, 15, 18, 22, 25, 29 and 33). Daratumumab (anti-CD38 antibody) (Myoderm Medical Supplies, 48 E Main St., Norristown, PA 19401) was purchased at the beginning of the study and stored at 4° C. according to manufacturer’s instructions. Final dose daratumumab received was 2.5 mg/kg, 7.5 mg/kg, and 20 mg/kg based on average body weight. Daratumumab was administered intraperitoneally (IP) using a 0.1 mL dosing volume, on a BIW schedule for 5 weeks (Days 1, 4, 8, 11, 15, 18, 22, 25, 29 and 33).

Tumors were measured twice weekly using Vernier calipers. Tumor volumes were calculated using standard equation: V = W² × L/2, where V = volume, W = width, and L = length for the tumor. When mean tumor volumes reached approximately 100 mm³, mice were randomized into control and treatment groups. Mice were randomized into 8 groups (n=8/group) and dosed with vehicle (20% HPβCD), I-263a, daratumumab, or the combination of I-263a plus daratumumab at doses and schedules as described above. Tumor size and body weight were measured twice a week for the duration of the study. Mice were euthanized when their tumor volumes exceeded approximately 2000 mm³ or when an individual tumor exceeded the humane end-point for size (the length of the tumor exceeded 2 cm).

In the LP-1 xenograft human multiple myeloma subcutaneous tumor model, mice were inoculated, randomized on Day 0 (18 days post inoculation), and treatments began on Day 1 for all groups.

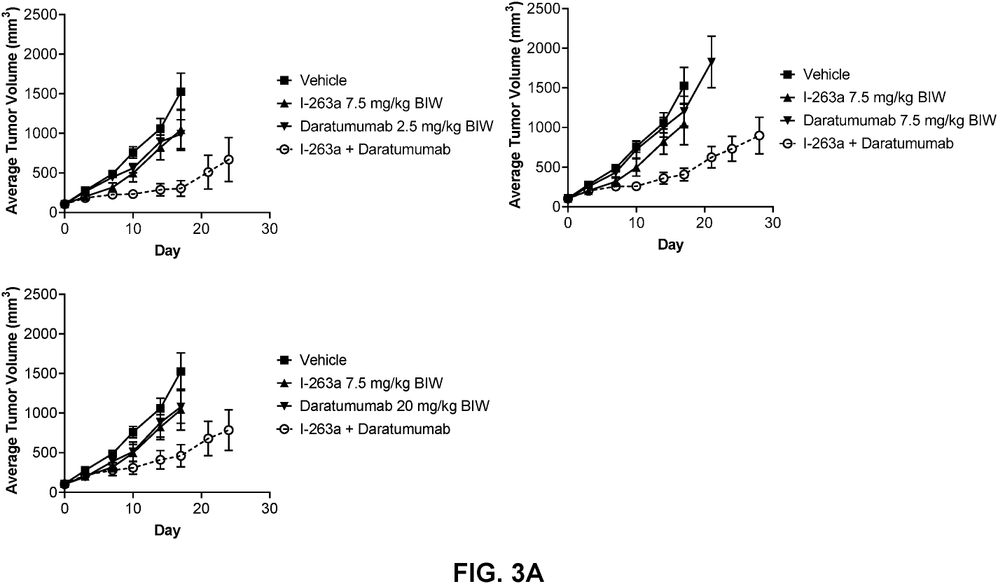

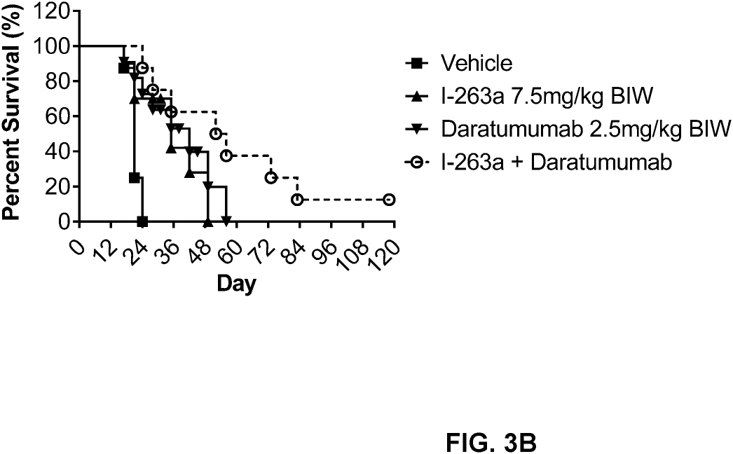

I-263a was tested at 7.5 mg/kg administered IV on a BIW (twice per week) schedule for five weeks (Days 1, 4, 8, 11, 15, 18, 22, 25, 29 and 33). Daratumumab was tested at 2.5 mg/kg, 7.5 mg/kg, or 20 mg/kg administered IP on a BIW schedule for five weeks (Days 1, 4, 8, 11, 15, 18, 22, 25, 29 and 33). In the combination treatment group, I-263a was administered first, followed immediately by the administration of daratumumab. One group served as the vehicle-treated group (Group 1) which received IV treatment with the vehicle for I-263a (20% HPβCD) on a BIW schedule for five weeks (Days 1, 4, 8, 11, 15, 18, 22, 25, 29 and 33). Average tumor growth curves are shown in FIG. 3A. Kaplan-Meier survival plots are shown in FIG. 3B.

In a pairwise comparison with vehicle, single agent treatment with I-263a at 7.5 mg/kg BIW for 5 weeks resulted in significant tumor growth inhibition (p < 0.01). Single agent treatment with daratumumab at 2.5 mg/kg or 7.5 mg/kg resulted in tumor growth curves not significantly different from vehicle treated mice (p > 0.05) whereas treatment with daratumumab at 20 mg/kg resulted in significant tumor growth inhibition (p < 0.01). Combining I-263a at 7.5 mg/kg BIW with daratumumab at 2.5 mg/kg, 7.5 mg/kg or 20 mg/kg resulted in significant tumor growth inhibition as compared to vehicle treated mice (p < 0.001). In a combination synergy analysis I-263a at 7.5 mg/kg BIW plus daratumumab at 2.5 mg/kg BIW or at 7.5 mg/kg BIW each resulted in synergistic combination benefit (p < 0.05) whereas I-263a at 7.5 mg/kg BIW plus daratumumab at 20 mg/kg BIW resulted in additive combination activity (p > 0.05), when compared to single agent treatments. The combination of I-263a with daratumumab at 2.5 mg/kg resulted in significantly improved survival relative to either single agent treatment (p < 0.01). P values for survival analysis were calculated by Weibull regression analysis.

The treatment groups from Study 3 are shown in Table 3a. The combination effect for the treatment period is shown in Table 3b.

TABLE 3a

Combination of Compound I-263a and daratumumab in a LP-1 xenograft model

| Study Group | Treatment | Dosing Regimen | Route | Tumor volume on Day 17 (± SEM) | GRI % | P-value |
|---|---|---|---|---|---|---|
| A | 20% HPβCD | BIW | IV | 1526 ± 233.7 | NA | NA |
| B | I-263a 7.5 mg/kg | BIW | IV | 1043.3 ± 260.1 | 15 | 0.13 |
| C | Daratumumab 2.5 mg/kg | BIW | IP | 988.8 ± 183.4 | 16 | 0.057 |
| D | Daratumumab 7.5 mg/kg | BIW | IP | 1204.8 ± 190.1 | 8 | 0.403 |
| E | Daratumumab 20 mg/kg | BIW | IP | 1078 ± 207.7 | 10 | 0.302 |
| F | I-263a 7.5 mg/kg, Daratumumab 2.5 mg/kg | BIW, BIW | IV, IP | 305.4 ± 97.6 | 74 | < 0.001 |
| G | I-263a 7.5 mg/kg, Daratumumab 7.5 mg/kg | BIW, BIW | IV, IP | 407 ± 78.7 | 58 | < 0.001 |
| H | I-263a 7.5 mg/kg, Daratumumab 20 mg/kg | BIW, BIW | IV, IP | 460.4 ± 141.2 | 58 | < 0.01 (0.004) |

TABLE 3b

Classification for in vivo combination of Compound I-263a and daratumumab in the LP-1 xenograft model

| Treatment | Synergy score | SEM | P-value | Classification |
|---|---|---|---|---|
| I-263a 7.5 mg/kg, Daratumumab 2.5 mg/kg | -43 | 16 | < 0.05 (0.011) | Synergistic |
| I-263a 7.5 mg/kg, Daratumumab 7.5 mg/kg | -36 | 16 | < 0.05 (0.033) | Synergistic |
| I-263a 7.5 mg/kg, Daratumumab 20 mg/kg | -33 | 18 | 0.085 | Additive |

### Study 4

MOLP-8 is a human multiple myeloma cell line. A MOLP-8 human xenograft tumor model was generated by subcutaneous inoculation with 4.0 × 10⁶ MOLP-8 cells (cell suspension) in 14 weeks old female SCID mice (Vital River Laboratory Animal Technology Co., Ltd, Beijing, China) in the flank. When the mean tumor volume reached approximately 170 mm³, the animals were randomized into one vehicle control and seven treatment groups (n=8/group). Mice were then dosed with 20% HPβCD or Compound I-263a or daratumumab over a 14-day period. Tumor growth and body weight were measured twice per week during the treatment and post-treatment periods, and mice were humanely euthanized once they had reached their humane endpoint.

A 0.75 mg/mL stock solution of I-263a was formulated weekly in 20% HPβCD and administered IV based on exact animal body weight on each day of treatment, using a dosing volume of 10 mL/kg body weight. Final dose I-263a received was 7.5 mg/kg. I-263a was administered on a BIW schedule for 2 weeks (Days 1, 4, 8, and 11). Daratumumab (anti-CD38 antibody) (Janssen Biotech, Inc., Horsham, PA) was stored at 4° C. according to manufacturer’s instructions. Final dose daratumumab received was 2.5 mg/kg, 7.5 mg/kg, and 20 mg/kg based on exact animal body weight on each day of treatment, using a dosing volume of 10 mL/kg body weight. Daratumumab was administered intraperitoneally (IP) using a 0.1 mL dosing volume, on a BIW schedule for 2 weeks (Days 1, 4, 8, and 11).

Tumors were measured twice weekly using Vernier calipers. Tumor volumes were calculated using standard equation: V = W² × L/2, where V = volume, W = width, and L = length for the tumor. When mean tumor volumes reached approximately 170 mm³, mice were randomized into control and treatment groups. Mice were randomized into 8 groups (n=8/group) and dosed with vehicle (20% HPβCD), I-263a, daratumumab, or the combination of I-263a plus daratumumab at doses and schedules as described above. Tumor size and body weight were measured twice a week for the duration of the study. Mice were euthanized when their tumor volumes exceeded approximately 2000 mm³ or when an individual tumor exceeded the humane end-point for size (the length of the tumor exceeded 2 cm).

In the MOLP-8 xenograft human multiple myeloma subcutaneous tumor model, mice were inoculated, randomized on Day 0 (10 days post inoculation), and treatments began on Day 1 for all groups.

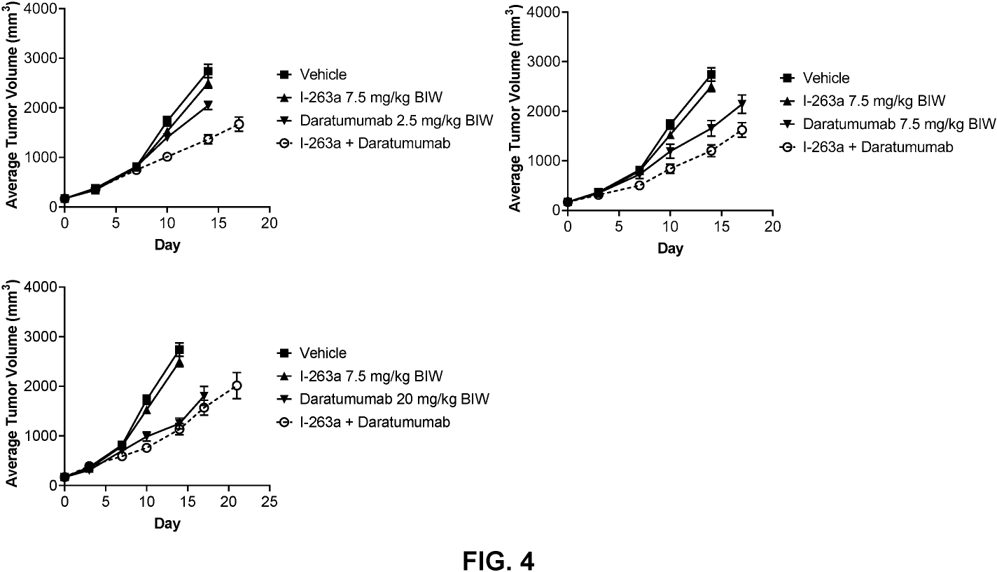

I-263a was tested at 7.5 mg/kg administered IV on a BIW (twice per week) schedule for 2 weeks (Days 1, 4, 8 and 11). Daratumumab was tested at 2.5 mg/kg, 7.5 mg/kg, or 20 mg/kg administered IP on a BIW schedule for two weeks (Days 1, 4, 8, and 11). In the combination treatment group, I-263a was administered first, followed immediately by the administration of daratumumab. One group served as the vehicle-treated group (Group 1) which received IV treatment with the vehicle for I-263a (20% HPβCD) on a BIW schedule for two weeks (Days 1, 4, 8, and 11). Average tumor growth curves are shown in FIG. 4.

Single agent treatment with I-263a at 7.5 mg/kg BIW for 2 weeks resulted in tumor growth curves not significantly different in a pairwise comparison with vehicle (p < 0.05). Single agent treatment with daratumumab at 2.5 mg/kg, 7.5 mg/kg or 20 mg/kg resulted in significantly inhibited growth curves as compared to vehicle (p < 0.01; p < 0.01; p < 0.001, respectively). Combining I-263a at 7.5 mg/kg BIW with daratumumab at 2.5 mg/kg, 7.5 mg/kg or 20 mg/kg resulted in significant tumor growth inhibition as compared to vehicle treated mice (p < 0.001). In a combination synergy analysis I-263a at 7.5 mg/kg BIW plus daratumumab at 2.5 mg/kg BIW resulted in synergistic combination benefit (p < 0.05) whereas I-263a at 7.5 mg/kg BIW plus daratumumab at 7.5 mg/kg or 20 mg/kg BIW each resulted in additive combination activity (p > 0.05), when compared to single agent treatments.

The treatment groups from Study 4 are shown in Table 4a. The combination effect for the treatment period is shown in Table 4b.

TABLE 4a

Combination of Compound I-263a and daratumumab in a MOLP-8 xenograft model

| Study Group | Treatment | Dosing Regimen | Route | Tumor volume on Day 14 (± SEM) | GRI% | P-value |
|---|---|---|---|---|---|---|
| A | 20% HPβCD | BIW | IV | 2742.3 ± 133.9 | NA | NA |
| B | I-263a 7.5 mg/kg | BIW | IV | 2500.8 ± 106.6 | 3 | 0.432 |
| C | Daratumumab 2.5 mg/kg | BIW | IP | 2047.4 ± 83.4 | 11 | < 0.05 (0.013) |
| D | Daratumumab 7.5 mg/kg | BIW | IP | 1653 ± 158.5 | 20 | < 0.01 (0.001) |
| E | Daratumumab 20 mg/kg | BIW | IP | 1250.6 ± 108.6 | 28 | < 0.001 |
| F | I-263a 7.5 mg/kg, Daratumumab 2.5 mg/kg | BIW, BIW | IV, IP | 1364 ± 91.5 | 27 | < 0.001 |
| G | I-263a 7.5 mg/kg, Daratumumab 7.5 mg/kg | BIW, BIW | IV, IP | 1204.6 ± 121.8 | 32 | < 0.001 |
| H | I-263a 7.5 mg/kg, Daratumumab 20 mg/kg | BIW, BIW | IV, IP | 1135.4 ± 112.7 | 38 | < 0.001 |

TABLE 4b

Classification for in vivo combination of Compound I-263a and daratumumab in the MOLP-8 xenograft model

| | | | | |
|---|---|---|---|---|
| Treatment | Synergy score | SEM | P-value | Classification |
| I-263a 7.5 mg/kg, Daratumumab 2.5 mg/kg | -13 | 5 | < 0.05 (0.015) | Synergistic |
| I-263a 7.5 mg/kg, Daratumumab 7.5 mg/kg | -9 | 6 | 0.116 | Additive |
| I-263a 7.5 mg/kg, | -7 | 6 | 0.222 | Additive |
| Daratumumab 20 mg/kg | | | | |

### Study 5

NCI-H929 is a human multiple myeloma cell line. A NCI-H929 human xenograft tumor model was generated by subcutaneous inoculation with 1.0 × 10⁷ NCI-H929 cells (cell suspension) in 7 weeks old female SCID mice (Vital River Laboratory Animal Technology Co., Ltd, Beijing, China) in the flank. When the mean tumor volume reached approximately 180 mm³, the animals were randomized into one vehicle control and seven treatment groups (n=10/group). Mice were then dosed with 20% HPβCD or Compound I-263a or daratumumab over a 14-day period. Tumor growth and body weight were measured twice per week during the treatment and post-treatment periods, and mice were humanely euthanized once they had reached their humane endpoint.

A 1.5 mg/mL stock solution of I-263a was formulated weekly in 20% HPβCD and administered IV based on exact animal body weight on each day of treatment, using a dosing volume of 5 mL/kg body weight. Final dose I-263a received was 7.5 mg/kg. I-263a was administered on a BIW schedule for 2 weeks (Days 0, 3, 7, and 10). Daratumumab (anti-CD38 antibody) (Janssen Biotech, Inc., Horsham, PA) was stored at 4° C. according to manufacturer’s instructions. Final dose daratumumab received was 2.5 mg/kg, 7.5 mg/kg, and 20 mg/kg based on average body weight. Daratumumab was administered intraperitoneally (IP) using a 0.1 mL dosing volume, on a BIW schedule for 2 weeks (Days 0, 3, 7, and 10).

Tumors were measured twice weekly using Vernier calipers. Tumor volumes were calculated using standard equation: V = W² × L/2, where V = volume, W = width, and L = length for the tumor. When mean tumor volumes reached approximately 180 mm³, mice were randomized into control and treatment groups. Mice were randomized into 8 groups (n=8/group) and dosed with vehicle (20% HPβCD), I-263a, daratumumab, or the combination of I-263a plus daratumumab at doses and schedules as described above. Tumor size and body weight were measured twice a week for the duration of the study. Mice were euthanized when their tumor volumes exceeded approximately 2000 mm³ or when an individual tumor exceeded the humane end-point for size (the length of the tumor exceeded 2 cm).

In the NCI-H929 xenograft human multiple myeloma subcutaneous tumor model, mice were inoculated, randomized on Day 0 (19 days post inoculation), and treatments began on Day 0 for all groups.

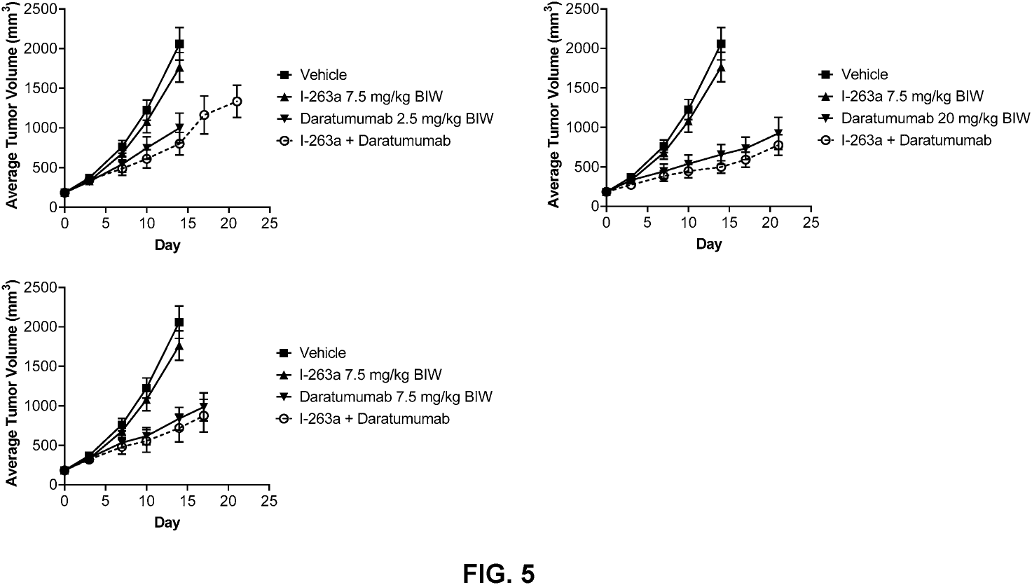

I-263a was tested at 7.5 mg/kg administered IV on a BIW (twice per week) schedule for 2 weeks (Days 0, 3, 7, and 10). Daratumumab was tested at 2.5 mg/kg, 7.5 mg/kg, or 20 mg/kg administered IP on a BIW schedule for two weeks (Days 0, 3, 7, and 10). In the combination treatment group, I-263a was administered first, followed immediately by the administration of daratumumab. One group served as the vehicle-treated group (Group 1) which received IV treatment with the vehicle for I-263a (20% HPβCD) on a BIW schedule for two weeks (Days 0, 3, 7, and 10). Average tumor growth curves are shown in FIG. 5.

Single agent treatment with I-263a at 7.5 mg/kg BIW for 2 weeks resulted in tumor growth curves not significantly different from vehicle (p > 0.05). Single agent treatment with daratumumab at 2.5 mg/kg, 7.5 mg/kg, or 20 mg/kg BIW for 2 weeks or when combined with I-263a significantly inhibited tumor growth curves in a pairwise comparison with vehicle (p < 0.001). In a combination synergy analysis, I-263a at 7.5 mg/kg BIW for 2 weeks plus daratumumab at 2.5 mg/kg, 7.5 mg/kg or 20 mg/kg BIW for 2 weeks each resulted in additive combination activity (p > 0.05).

The treatment groups from Study 5 are shown in Table 5a. The combination effect for the treatment period is shown in Table 5b.

TABLE 5a

Combination of Compound I-263a and daratumumab in a NCI-H929 xenograft model

| Study Group | Treatment | Dosing Regimen | Route | Tumor volume on Day 14 (± SEM) | GRI% | P-value |
|---|---|---|---|---|---|---|
| A | 20% HPβCD | BIW | IV | 2059 ± 206.5 | NA | NA |
| B | I-263a 7.5 mg/kg | BIW | IV | 1763.8 ± 187.6 | 6 | 0.225 |
| C | Daratumumab 2.5 mg/kg | BIW | IP | 999.5 ± 187.7 | 37 | < 0.001 |
| D | Daratumumab 7.5 mg/kg | BIW | IP | 838.3 ± 142.8 | 45 | < 0.001 |
| E | Daratumumab 20 mg/kg | BIW | IP | 656.2 ± 127.2 | 54 | < 0.001 |
| F | I-263a 7.5 mg/kg, Daratumumab 2.5 mg/kg | BIW, BIW | IV, IP | 801.4 ± 142.6 | 46 | < 0.001 |
| G | I-263a 7.5 mg/kg, Daratumumab 7.5 mg/kg | BIW, BIW | IV, IP | 718.8 ± 174.5 | 51 | < 0.001 |
| H | I-263a 7.5 mg/kg, Daratumumab 20 mg/kg | BIW, BIW | IV, IP | 497.6 ± 78.8 | 62 | < 0.001 |

TABLE 5b

Classification for in vivo combination of Compound I-263a and daratumumab in the NCI-H929 xenograft model

| Treatment | Synergy score | SEM | P-value | Classification |
|---|---|---|---|---|
| I-263a 7.5 mg/kg, Daratumumab 2.5 mg/kg | -3 | 10 | 0.741 | Additive |
| I-263a 7.5 mg/kg, Daratumumab 7.5 mg/kg | 0 | 9 | 0.956 | Additive |
| I-263a 7.5 mg/kg, Daratumumab 20 mg/kg | -3 | 8 | 0.732 | Additive |

### Study 6

RPMI-8226 is a human multiple myeloma cell line. A RPMI-8226 human xenograft tumor model was generated by subcutaneous inoculation with 1.0 × 10⁷ NCI-H929 cells (cell suspension) in 7 weeks old female SCID mice (Vital River Laboratory Animal Technology Co., Ltd, Beijing, China) in the flank. When the mean tumor volume reached approximately 180 mm³, the animals were randomized into one vehicle control and seven treatment groups (n=10/group). Mice were then dosed with 20% HPβCD or Compound I-263a or daratumumab over a 14-day period. Tumor growth and body weight were measured twice per week during the treatment and post-treatment periods, and mice were humanely euthanized once they had reached their humane endpoint.

A 1.5 mg/mL stock solution of I-263a was formulated weekly in 20% HPβCD and administered IV based on exact animal body weight on each day of treatment, using a dosing volume of 5 mL/kg body weight. Final dose I-263a received was 7.5 mg/kg. I-263a was administered on a BIW schedule for 2 weeks (Days 0, 3, 7, and 10). Daratumumab (anti-CD38 antibody) (Janssen Biotech, Inc., Horsham, PA) was stored at 4° C. according to manufacturer’s instructions. Final dose daratumumab received was 2.5 mg/kg, 7.5 mg/kg, and 20 mg/kg based on average body weight. Daratumumab was administered intraperitoneally (IP) using a 0.1 mL dosing volume, on a BIW schedule for 2 weeks (Days 0, 3, 7, and 10).

Tumors were measured twice weekly using Vernier calipers. Tumor volumes were calculated using standard equation: V = W² × L/2, where V = volume, W = width, and L = length for the tumor. When mean tumor volumes reached approximately 200 mm³, mice were randomized into control and treatment groups. Mice were randomized into 8 groups (n=8/group) and dosed with vehicle (20% HPβCD), I-263a, daratumumab, or the combination of I-263a plus daratumumab at doses and schedules as described above. Tumor size and body weight were measured twice a week for the duration of the study. Mice were euthanized when their tumor volumes exceeded approximately 2000 mm³ or when an individual tumor exceeded the humane end-point for size (the length of the tumor exceeded 2 cm).

In the RPMI-8226 human multiple myeloma subcutaneous tumor model, mice were inoculated, randomized on Day 0 (29 days post inoculation), and treatments began on Day 0 for all groups.

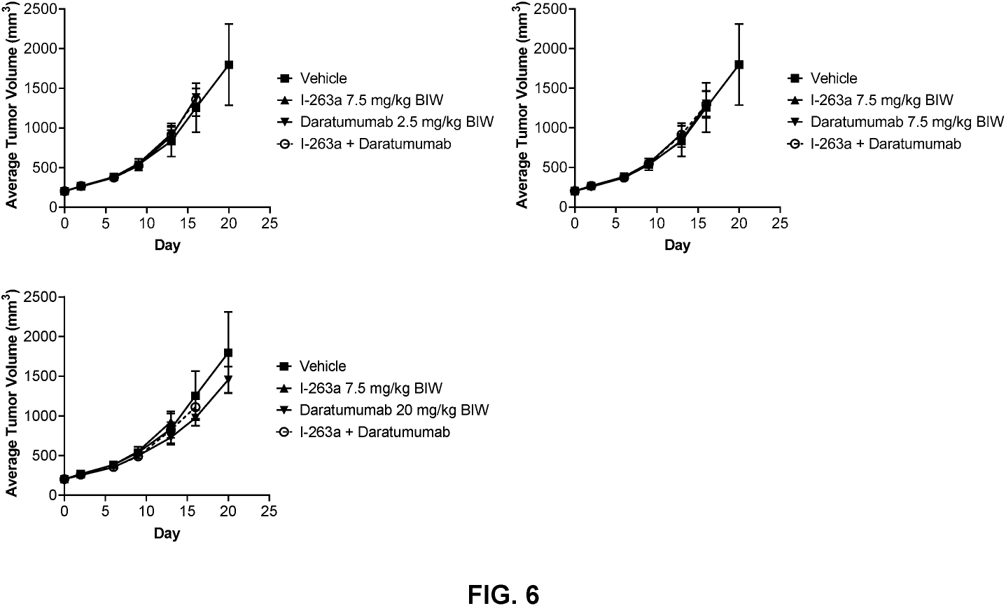

I-263a was tested at 7.5 mg/kg administered IV on a BIW (twice per week) schedule for 2 weeks (Days 0, 3, 7, and 10). Daratumumab was tested at 2.5 mg/kg, 7.5 mg/kg, or 20 mg/kg administered IP on a BIW schedule for two weeks (Days 0, 3, 7, and 10). In the combination treatment group, I-263a was administered first, followed immediately by the administration of daratumumab. One group served as the vehicle-treated group (Group 1) which received IV treatment with the vehicle for I-263a (20% HPβCD) on a BIW schedule for two weeks (Days 0, 3, 7, and 10). Average tumor growth curves are shown in FIG. 6.

In a pairwise comparison with vehicle, single agent treatment with I-263a at 7.5 mg/kg BIW for 2 weeks or daratumumab at 2.5 mg/kg, 7.5 mg/kg, or 20 mg/kg BIW for 2 weeks, or the combination of both agents resulted in tumor growth curves not significantly different than vehicle-treated tumors (p > 0.05). In a combination synergy analysis, I-263a at 7.5 mg/kg BIW for 2 weeks plus daratumumab at 2.5 mg/kg, 7.5 mg/kg or 20 mg/kg BIW for 2 weeks each resulted in additive combination activity when compared to single-agent treatments (p > 0.05).

The treatment groups from Study 6 are shown in Table 6a. The combination effect for the treatment period is shown in Table 6b.

TABLE 6a

Combination of Compound I-263a and daratumumab in a RPMI-8226 xenograft model

| Study Group | Treatment | Dosing Regimen | Route | Tumor volume on Day 20 (± SEM) | GRI% | P-value |
|---|---|---|---|---|---|---|
| A | 20% HPβCD | BIW | IV | 1798.2 ± 161.6 | NA | NA |
| B | I-263a 7.5 mg/kg | BIW | IV | 1763.2 ± 213.2 | -12 | 0.214 |
| C | Daratumumab 2.5 mg/kg | BIW | IP | 1875.5 ± 170.1 | -6 | 0.429 |
| D | Daratumumab 7.5 mg/kg | BIW | IP | 1510.7 ± 144.6 | -2 | 0.857 |
| E | Daratumumab 20 mg/kg | BIW | IP | 1456.6 ± 166.6 | 11 | 0.15 |
| F | I-263a 7.5 mg/kg, Daratumumab 2.5 mg/kg | BIW, BIW | IV, IP | 1482.3 ± 118.5 | -2 | 0.795 |
| G | I-263a 7.5 mg/kg, Daratumumab 7.5 mg/kg | BIW, BIW | IV, IP | 1459.1 ± 178.5 | 0 | 0.985 |
| H | I-263a 7.5 mg/kg, Daratumumab 20 mg/kg | BIW, BIW | IV, IP | 1624.5 ± 228.2 | 7 | 0.52 |

TABLE 6b

Classification for in vivo combination of Compound I-263a and daratumumab in the RPMI-8226 xenograft model

| Treatment | Synergy score | SEM | P-value | Classification |
|---|---|---|---|---|
| I-263a 7.5 mg/kg, Daratumumab 2.5 mg/kg | -15 | 13 | 0.233 | Additive |
| I-263a 7.5 mg/kg, Daratumumab 7.5 mg/kg | -14 | 14 | 0.358 | Additive |
| I-263a 7.5 mg/kg, Daratumumab 20 mg/kg | -7 | 14 | 0.599 | Additive |

### Study 7

Daudi is a human Burkitt’s Lymphoma cell line. A Daudi human xenograft tumor model was generated by subcutaneous inoculation with 2.0 × 106 Daudi cells (cell suspension) in 7 weeks old female CB17 SCID mice (Charles River Laboratories, 251 Ballardvale St., Wilmington, MA) in the flank. When the mean tumor volume reached approximately 110 mm3, the animals were randomized into one vehicle control and six treatment groups (n=8/group). Mice were then dosed with 20% HPβCD or Compound I-263a and/or daratumumab or AB79 over a 21-day period. Tumor growth and body weight were measured twice per week during the treatment and post-treatment periods, and mice were humanely euthanized once they had reached their humane endpoint.

A 0.75 mg/mL stock solution of I-263a was formulated weekly in 20% HPβCD and administered intravenously (IV) based on an average body weight using a 0.2 mL dosing volume. Final doses of I-263a received were 7.5 mg/kg. I-263a was administered on a BIW schedule for 3 weeks (Days 1, 4, 8, 11, 15, and 18). Daratumumab (anti-CD38 antibody) (Myoderm Medical Supplies, 48 E Main St., Norristown, PA 19401) was purchased at the beginning of the study and stored at 4° C. according to manufacturer’s instructions. Final dose of daratumumab received was 7.5 mg/kg based on average body weight. Daratumumab was administered intraperitoneally (IP) using a 0.1 mL dosing volume, on a BIW schedule for 3 weeks (Days, 1, 4, 8, 11, 15, and 18). AB79 was formulated on the day of dosing in 0.9% saline and administered intraperitoneally (IP) using a 0.1 mL dosing volume, on a BIW schedule for 3 weeks (Days, 1, 4, 8, 11, 15, and 18). Final dose of AB79 was 7.5 mg/kg based on average body weight.

Tumors were measured twice weekly using Vernier calipers. Tumor volumes were calculated using standard equation: V = W2 × L/2, where V = volume, W = width, and L = length for the tumor. When mean tumor volumes reached approximately 110 mm³, mice were randomized into control and six treatment groups (n=8/group), and dosed with vehicle (20% HPβCD), I-263a, daratumumab, AB79, or the combination of I-263a plus daratumumab or AB79 at doses and schedules described above. Tumor size and body weight were measured twice a week for the duration of the study. Mice were euthanized when their tumor volumes exceeded approximately 2000 mm3 or when an individual tumor exceeded the humane end-point for size (the length of the tumor exceeded 2 cm).

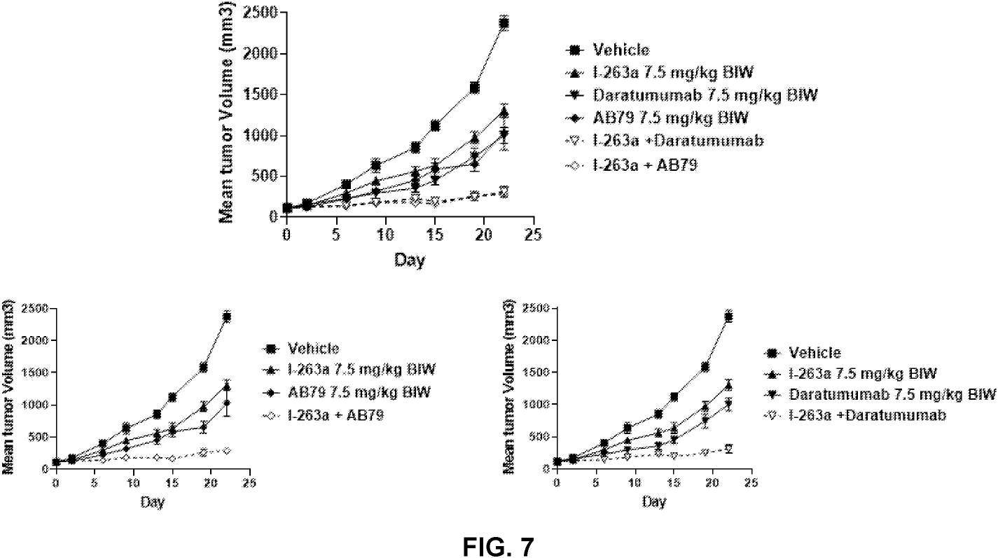

In the Daudi xenograft human Burkitt’s lymphoma subcutaneous tumor model, mice were inoculated, randomized on Day 0 (17 days post inoculation), and treatments began on Day 1 for all groups. I-263a was tested at 7.5 mg/kg administered IV on a BIW (twice per week) schedule for three weeks (Days 1, 4, 8, 11, 15, and 18). Daratumumab was tested at 7.5 mg/kg administered IP on a BIW schedule for three weeks (Days 1, 4, 8, 11, 15 and 18), and AB79 was tested at 7.5 mg/kg administered IP on a BIW schedule for three weeks (Days 1, 4, 8, 11, 15 and 18). In the combination treatment group, I-263a was administered first, followed immediately by the administration of daratumumab or AB79. One group served as the vehicle-treated group (Group 1) which received IV treatment with the vehicle for I-263a (20% HPβCD) on a BIW schedule for three weeks (Days 1, 4, 8, 11, 15, and 18). Statistical analyses were performed on data up to and including Day 22. Average tumor growth curves are shown in FIG. 7.

In a pairwise comparison with vehicle, single agent treatment with I-263a at 7.5 mg/kg BIW resulted in tumor growth inhibition relative to vehicle-treated tumors (p = 0.105); single agent treatment with daratumumab at 7.5 mg/kg BIW resulted in tumor growth inhibition relative to vehicle-treated tumors (p < 0.001); single agent treatment with AB79 at 7.5 mg/kg BIW resulted in tumor growth inhibition relative to vehicle-treated tumors (p = 0.002); the combination of both I-263a and daratumumab agents resulted in tumor growth inhibition relative to vehicle-treated tumors (p < 0.001), or the combination of both I-263a and AB79 agents resulted in tumor growth inhibition relative to vehicle-treated tumors (p < 0.001). In a combination synergy analysis, I-263a at 7.5 mg/kg BIW plus daratumumab at 7.5 mg/kg BIW resulted in synergistic combination activity when compared to single-agent treatments (p = 0.029); or I-263a at 7.5 mg/kg BIW plus AB79 at 7.5 mg/kg BIW resulted in synergistic combination activity when compared to single-agent treatments (p = 0.009). These results demonstrate I-263a moderately inhibits tumor growth in a Daudi human Burkitt’s Lymphoma mouse model and offers synergistic combination benefit when combined with daratumumab or AB79.

The treatment groups from Study 7 are shown in Table 7a. The combination effect for the treatment period is shown in Table 7b.

TABLE 7a

Combination of Compound I-263a and Daratumumab or AB79 in the Daudi Xenograft Model

| Study Group | Treatment | Dosing Regiment | Route | Tumor Volume on Day 22 (±SEM) | GRI% | P-value |
|---|---|---|---|---|---|---|
| A | 20% HPβCD | BIW | IV | 2377.5 (±91.1) | | |
| B | I-263a 7.5 mg/kg | BIW | IV | 1305 (±83.6) | 13 | 0.105 |
| C | Daratumumab 7.5 mg/kg | BIW | IP | 1004.9 (±97.1) | 31 | < 0.001 |
| D | AB79 7.5 mg/kg | BIW | IP | 1033.8 (±208.8) | 29 | 0.002 |
| E | I-263a 7.5 mg/kg Daratumumab 7.5 mg/kg | BIW BIW | IV IP | 314.1 (±64.3) | 72 | < 0.001 |
| F | I-263a 7.5 mg/kg AB79 7.5 mg/kg | BIW BIW | IV IP | 285.5 (±51.8) | 77 | < 0.001 |

TABLE 7b

Classification for in vivo Combination of Compound I-263a and Daratumumab or AB79 in the Daudi Xenograft Model

| Treatment | Synergy score | SEM (%) | P-value | Classification |
|---|---|---|---|---|
| I-263a 7.5 mg/kg, Daratumumab 7.5 mg/kg | -28 | 12 | 0.029 | Synergistic |
| I-263a 7.5 mg/kg, AB79 7.5 mg/kg | -35 | 12 | 0.009 | Synergistic |

### Example 2: Clinical Study Evaluating Compound I-263a in Combination With an Anti-CD38 Monoclonal Antibodies in Treatment of Patients With Relapsed an/or Refractory Multiple Myeloma

A Phase 1b/2, open-label, multicenter, dose escalation study will be conducted to investigating the combination of Compound I-263a and monoclonal antibodies (mAbs) in adult patients with relapsed and/or refractory multiple myeloma (RRMM). The study will be conducted in two phases: 1) Phase 1b dose escalation of Compound I-263a guided by Bayesian Optimal Interval Design with Informative Prior (iBOIN) in combination with fixed doses of mezagitamab or daratumumab and hyaluronidase-fihj, respectively in patients with RRMM; and 2) Phase 2 study of Compound I-263a-based mAb combination in patients with RRMM.

Treatment cycle duration is 28 days. Compound I-263a in combination with the mAbs will be administered for up to 24 cycles or until disease progression or unacceptable toxicity, whichever comes first. Patients with demonstrated clinical benefit may continue treatment beyond 24 cycles with the agreement of the sponsor/designee.

Patient participation will include a screening phase, a treatment phase, and a follow-up phase. The screening phase will be up to approximately 28 days before Cycle 1, Day 1 (C1D1). The treatment phase will extend from C1D1 until patients experience disease progression, unacceptable toxicity, until any other discontinuation criterion is met, or to a maximum of 24 cycles. The follow-up phase of the study begins once a patient discontinues study treatment and completes the end-of-treatment (EOT) visit; study follow-up continues until the study ends or the patient completes overall survival (OS) follow-up.

Overall this study will enroll up to approximately 81 patients in North America and/or globally. Approximately 15 patients will be enrolled in each dosing schedule in Phase 1b, Part 1 and in Phase 1b, Part 2. Up to approximately 36 patients will be enrolled in Phase 2. Approximately 15 sites will participate in this study.

### Phase 1b Study Design

The Phase 1b part of the study will enroll patients with RRMM with the purpose of defining the recommended phase 2 dose (RP2D) and schedule of Compound I-263a in combination with either mezagitamab or daratumumab and hyaluronidase-fihj, respectively. The first part of the Phase 1b study will determine the dose and schedule of Compound I-263a in combination with a fixed dose and schedule of mezagitamab for expansion in Phase 2. The second part of the Phase 1b study will confirm the dose of Compound I-263a for the combination with daratumumab and hyaluronidase-fihj.

### Phase 1b Part 1

Dose escalation of Compound I-263a will be guided by a iBOIN. Up to approximately 15 patients will be enrolled to each of the Compound I-263a schedules until either a maximum tolerated dose (MTD) or a pharmacologically active dose (PAD) is identified:
Arm A: Compound I-263a is given intravenously (IV) twice weekly (BIW) on Days 1, 4, 8, 11, and 15 in Cycles 1 and 2 followed by every 2 weeks in Cycles 3 through 6, then monthly. Compound I-263a will be given in combination with mezagitamab.
Arm B: Compound I-263a is given IV weekly (QW) on Days 1, 8, 15, and 22 in Cycles 1 and 2 followed by every 2 weeks in Cycles 3 through 6, then monthly. Compound I-263a will be given in combination with mezagitamab.

The starting dose for Compound I-263a will be 60 mg. The dose of mezagitamab is the established RP2D at 600 mg. Compound I-263a will be administered as a 60 ±10 minute IV infusion.

Mezagitamab will be dosed 600 mg SC weekly in Cycles 1 and 2, followed by every 2 weeks in Cycles 3 through 6, then monthly.

Once enrolled into the study, patients will be assigned to a treatment arm in a nonrandomized, sequential manner based upon the recruitment status of the Compound I-263a arm schedule, as communicated by the sponsor/ designee. A minimum of 3 patients will be enrolled in the first dose cohort. In the first dose cohort, patient enrollment will be staggered between the first and second patients by 7 days. The second and third patients can be dosed concurrently if the first patient in the cohort has gone through the Day 8 visit without clinically significant acute toxicities. Subsequent dose cohorts will not require staggering between patients.

Dose escalation will begin with the BIW schedule and will be followed by the QW schedule at the same starting dose level. Compound I-263a dose escalation will be evaluated separately for each of the Compound I-263a schedules.

Dose escalation decisions will be made by a Safety Monitoring Committee (SMC), composed of the principal investigators and sponsor. The SMC will regularly review safety data to ensure patients’ safety throughout the Phase 1b portion of the study. Dose escalation will follow an iBOIN design. Dose escalation decisions will take into consideration primarily the dose-limiting toxicities (DLTs) observed in Cycle 1 in the patients enrolled in each dose level/schedule according to DLT rules. Available safety information beyond Cycle 1, pharmacokinetic (PK), and pharmacodynamic information from previously dose patients will also be considered.

Evaluation of intermediate doses or doses up to that evaluated and found safe in a Compound I-263a monotherapy study, alternative dosing schedules (dosing interval), and expansion of an existing dose level are all permissible following agreement by the SMC, if such measures are needed for patient safety or for a better understanding of the dose-related toxicity, efficacy, exposure, or pharmacodynamics of Compound I-263a. The dose escalation/de-escalation rules based on iBOIN design will be considered as a guidance for the next dose level, however, the final decision on the dose will be made by the SMC.

The selection of an RP2D which includes the dose level and schedule of Compound I-263a in combination with mezagitamab will be made by the sponsor following evaluation of the available data from the Phase 1b, Part 1 portion of the trial which will include, but is not limited to safety data, preliminary PK data, preliminary pharmacodynamic data, preliminary translational data, PK/pharmacodynamic modeling and preliminary antitumor activity. The RP2D may not be higher than either the MTD as determined by iBOIN. Upon review of available phase 1 data and agreement on the RP2D by the SMC, the Phase 1b Part 2 portion of the study of the combination of Compound I-263a with daratumumab and hyaluronidase-fihj may begin.

### Phase 1b Part 2

In the second part of the Phase 1b study, the RP2D of Compound I-263a in the combination with daratumumab and hyaluronidase-fihj will be determined. Dose escalation of Compound I-263a will be guided by iBOIN. The starting dose will be one dose-level below the RP2D defined for the combination with mezagitamab in Phase 1b Part 1. The dose of daratumumab and hyaluronidase-fihj will be 1800 mg. Daratumumab and hyaluronidase-fihj will be dosed weekly in Cycles 1 and 2, followed by every 2 weeks in Cycles 3 through 6, then monthly. A minimum of 3 patients will be enrolled in the first cohort of this combination and up to approximately 15 patients will be enrolled for the dose escalation.

The selection of an RP2D of Compound I-263a in combination with daratumumab and hyaluronidase-fihj will be made by the sponsor following evaluation of the available data from the Phase 1b, Part 2 portion of the trial which will include, but is not limited to safety data, preliminary PK data, preliminary pharmacodynamic data, preliminary translational data, PK/pharmacodynamic modeling and preliminary antitumor activity. The RP2D may not be higher than the MTD as determined by iBOIN.

### Phase 1b Primary Endpoints

The primary endpoints for the phase 1b trial will include: frequency and severity of treatment-emergent adverse events (TEAEs) for all dose groups; and occurrence of DLT in Cycle 1.

### Phase 1b Secondary Endpoints

The secondary endpoints for the phase 1b trial will include: overall response rate (ORR), clinical benefit rate (CBR), duration of response (DOR), time to progression (TTP), time to next treatment (TTNT), progression-free survival (PFS), and overall survival (OS) according to standard International Myeloma Working Group (IMWG) criteria; and Compound I-263a-SUMO adduct formation and SUMO pathway inhibition in blood.

### Phase 2 Study Design

The Phase 2 portion of the study will explore the efficacy and safety of Compound I-263a in combination with an anti-CD38 antibody (mezagitamab or daratumumab and hyaluronidase-fihj) in patients with RRMM. Patients will be treated with the RP2D defined in Phase 1b for the respective antibody. Cycle duration is 28 days and treatment will be administered for up to 24 cycles or until disease progression or unacceptable toxicity, whichever occurs first.

Patients with RRMM will be treated with the RP2D of Compound I-263a with fixed doses of the anti-CD-38 mAb, mezagitamab or daratumumab and hyaluronidase-fihj.

An adaptive 2-stage design for a single proportion will be used in Phase 2. For Stage I, each cohort will be analyzed when a pre-specified number of patients have been enrolled and had the opportunity to complete 4 cycles of treatment. Enrollment may be paused until the Stage I analysis is completed. If the pre-specified minimal response rate is not achieved in the first stage, enrollment will be closed. If the required response rate during Stage I or a good clinical benefit rate (CBR) is observed as mentioned above, then additional patients will be enrolled in the second stage of the corresponding cohort until a predetermined number of additional patients has been reached. The final analysis of the primary endpoints will take place when all ongoing patients have had the opportunity complete the 12-month disease assessment.

The selection of the anti-CD38 antibody (mezagitamab or daratumumab and hyaluronidase-fihj) for combination with Compound I-263a in Phase 2 will be made by the sponsor following evaluation of the available data from the Phase 1b, Part 1 portion of the trial which will include, but is not limited to safety data, selected RP2D, frequency of dose reduction/discontinuation observed for anti-CD38 antibody, preliminary pharmacodynamic data, preliminary translational data, PK/pharmacodynamic modeling and preliminary antitumor activity.

### Phase 2 Primary Endpoints

The primary endpoints for the phase 2 trial will include: overall response rate (ORR) (response of at least partial response (PR)) based on investigator’s assessment according to standard International Myeloma Working Group (IMWG) disease response criteria.

### Phase 2 Secondary Endpoints

The secondary endpoints for the phase 2 trial will include: frequency and severity of TEAEs; CBR, DOR, TTP, TTNT, PFS, and OS based on IMWG criteria; percentage of participants with minimal residual disease (MRD) negative status as determined by next-generation sequencing (NGS); MRD negative rate at 1 year, defined as percentage of participants who have achieved MRD negative status at 1 year; and durable MRD negative rate, defined as the number of participants who have achieved MRD negative status (at 10^-5) at 2 bone marrow aspirate (BMA) examinations that are a minimum of 1 year apart, without any examination showing MRD positive status in between.

The trial will be conducted in conformance with Good Clinical Practices.

Having now fully described this invention, it will be understood by those of ordinary skill in the art that the same can be performed within a wide and equivalent range of conditions, formulations, and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are fully incorporated by reference herein in their entirety.

What is claimed is:

1. A method of treating a disorder, wherein the disorder is cancer or autoimmune disease, comprising administering to a patient in need of said treating a combination of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and an anti-CD38 antibody.

2. The method of claim 1, wherein the anti-CD38 antibody is selected from the group consisting of isatuximab, daratumumab, mezagitamab, MOR03087 (also known as MOR202), SG303, mAb024, and mAb003.

3. The method of any one of claims 1-2, wherein the anti-CD38 antibody is mezagitamab,.

4. The method of any one of claims 1-2, wherein the anti-CD38 antibody is daratumumab.

5. The method of claim 4, wherein the daratumumab is provided in an injectable formulation comprising daratumumab and hyaluronidase-fihj.

6. The method of any one of claims 1-5, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered orally.

7. The method of any one of claims 1-5, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered intravenously, or subcutaneously.

8. The method of any one of claims 1-5, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered by intravenous infusion.

9. The method of any one of claims 1-8, wherein the disorder is cancer.

10. The method of any one of claims 1-9, wherein the disorder is CD38 positive cancer.

11. The method of any one of claims 1-10, wherein the disorder is hematological malignancy.

12. The method of any one of claims 1-11, wherein the disorder is multiple myeloma.

13. The method of any one of claims 1-12, wherein the disorder is CD38 positive multiple myeloma.

14. The method of any one of claims 1-13, wherein the disorder is CD38 positive relapsed or refractory multiple myeloma.

15. The method of any one of claims 1-11, wherein the disorder is lymphoma or leukemia.

16. The method of any one of claims 1-11 and 15, wherein the disorder is follicular lymphoma (FL), mantle cell lymphoma (MCL), or Diffuse large B-cell lymphoma (DLBCL), or Burkitt lymphoma.

17. The method of any one of claims 1-16, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered once every two weeks, once every week, twice a week, three times a week, or daily.

18. The method of claim 1-17, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered once every week.

19. The method of claim 1-17, wherein the [(1R,2S,4R)-4-{[5-({4-[(1R)-7-chloro-l,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered twice every week.

20. The method of any one of claims 1-17, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered in a treatment cycle of 14 days, 21 days, or 28 days.

21. The method of any one of claims 1-17, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1, 4, 8, and 11 of a 14 days cycle.

22. The method of any one of claims 1-17, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 0, 3, 7, and 10 of a 14 day cycle.

23. The method of any one of claims 1-17, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1, 8, 15, and 22 of a 28 day cycle.

24. The method of any one of claims 1-17, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1, 8, 15, and 22 of a 28 day cycle in cycles 1 and 2 followed by every 2 weeks in cycles 3 through 6, then monthly.

25. The method of any one of claims 1-17, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1, 4, 8, 11, and 15 of a 28 day cycle.

26. The method of any one of claims 1-17, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1, 4, 8, 11, and 15 of a 28 day cycle in cycles 1 and 2 followed by every 2 weeks in cycles 3 through 6, then monthly.

27. The method of any one of claims 1-17, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1 and 15 of a 28 day cycle.

28. The method of any one of claims 1-17, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on day 1 of a 28 day cycle.

29. The method of any one of claims 1-17, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1, 4, 8, and 11 of a 21 day cycle.

30. The method of any one of claims 1-17, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1 and 8 of a 21 day cycle.

31. The method of any one of claims 1-30, wherein 40 mg, 60 mg, 90 mg, 120 mg, 160 mg, 200 mg, or 250 mg of the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered.

32. The method of any one of claims 1-30, wherein 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, or 250 mg of the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered.

33. The method of any one of claims 1-32, wherein the anti-CD38 antibody is administered once every four weeks, once every three weeks, once every two weeks, once every week, twice a week, three times a week, or daily.

34. The method of any one of claims 1-32, wherein the anti-CD38 antibody is administered once every week, once every two weeks, once every three weeks, or once every four weeks.

35. The method of any one of claims 1-30, wherein the anti-CD38 antibody is administered on days 1, 8, 15, and 22 of a 28 day cycle.

36. The method of any one of claims 1-30, wherein the anti-CD38 antibody is administered on days 1 and 15 of a 28 day cycle.

37. The method of any one of claims 1-30, wherein the anti-CD38 antibody is administered on day 1 of a 28 day cycle.

38. The method of any one of claims 1-30, wherein the anti-CD38 antibody is administered once weekly in cycles 1 and 2 of a 28-day cycle, followed by every 2 weeks in cycles 3 through 6, then monthly.

39. The method of any one of claims 1-30, wherein the anti-CD38 antibody is administered weekly in cycles 1 and 2, followed by every 2 weeks in cycles 3 through 6, then monthly.

40. The method of any one of claims 1-38, wherein 300 mg, 500 mg, 600 mg, 700 mg, 900 mg, 1100 mg, 1200 mg, or 1800 mg of the anti-CD38 antibody is administered.

41. A kit comprising a medicament for use in treating cancer or autoimmune disease in a subject in need of such treatment, wherein the kit comprises a medicament comprising an SAE inhibitor, and instructions for administering the SAE inhibitor and one or more anti-CD38 antibodies.

42. A kit comprising a medicament for use in treating cancer or autoimmune disease in a subject in need of such treatment, wherein the kit comprises a medicament comprising one or more anti-CD38 antibodies, and instructions for administering the one or more anti-CD38 antibodies and an SAE inhibitor.

43. A kit comprising a medicament for use in treating cancer or autoimmune disease in a subject in need of such treatment, wherein the kit comprises a medicament comprising an SAE inhibitor and a medicament comprising one or more anti-CD38 antibodies, and instructions for administering the SAE inhibitor and the one or more anti-CD38 antibodies.

44. The kit of any one of claims 41-43, wherein the kit further comprises one or more additional therapeutic agents.

45. A method of treating a disorder, wherein the disorder is cancer or autoimmune disease, comprising administering to a patient in need of said treating a combination of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, an anti-CD38 antibody, and an additional therapeutic agent.

46. The method of claim 45, wherein the additional therapeutic agent is lenalidomide, dexamethasone, bortezomib, melphalan, prednisone, pomalidomide, or combinations thereof.

47. The method of claim 45, wherein the additional therapeutic agent is lenalidomide and dexamethasone.

48. The method of claim 45, wherein the additional therapeutic agent is bortezomib.

49. The method of claim 45, wherein the additional therapeutic agent is melphalan and prednisone.

50. The method of claim 45, wherein the additional therapeutic agent is pomalidomide and dexamethasone.
